(19) United States
(12) Patent Application Publication
(10) Pub. No.: US 2002/0048792 A1
(43) Pub. Date: Apr. 25, 2002

(54) Methods and materials for regulated production of proteins

(75) Inventors: Sridaran Natesan, Chestnut Hill, MA (US); Timothy P. Clackson, Cambridge, MA (US); Roy M. Pollock, Medford, MA (US)

Correspondence Address: ARIAD Pharmaceuticals, Inc., 26 Landsdowne Street, Cambridge, MA, 02139, US

(73) Assignee: ARIAD Gene Therapeutics, Inc.

(21) Appl. No.: 09/906,189

(22) Filed: Jul. 16, 2001

Related U.S. Application Data
(63) Continuation of application No. 09/488,267, filed on Jan. 20, 2000, now abandoned.
Application No. 09/488,267 is a continuation-in-part of application No. 09/140,149, filed on Aug. 26, 1998, now Pat. No. 6,117,680.
Application No. 09/140,149 is a continuation-in-part of application No. 09/126,009, filed on Jul. 29, 1998, now abandoned.
Application No. 09/126,009 is a continuation-in-part of application No. 08/920,610, filed on Aug. 27, 1997, now Pat. No. 6,015,709.
Application No. 08/920,610 is a continuation-in-part of application No. 08/918,401, filed on Aug. 26, 1997, now abandoned.
Application No. 08/920,610 is a continuation-in-part of application No. PCT/US97/15219, filed on Aug. 27, 1997.

(51) Int. Cl. C12P 21/02; C12N 15/87

(52) U.S. Cl. 435/69.1; 435/455; 435/320.1

(57) ABSTRACT

This invention provides methods and materials for regulated production of proteins.

### RELATED APPLICATIONS

This application is a continuation of U.S. Ser. No. 09/488,267 filed Jan. 20, 2000, which is a continuation in part of U.S. Ser. No. 09/140,149 filed Aug. 26, 1998, which is a continuation in part of U.S. Ser. No. 09/126,009 filed Jul. 29, 1998, which is a continuation in part of 08/920,610 filed Aug. 27, 1997, now U.S. Pat. No. 6,015,709, which is a continuation in part of U.S. Ser. No. 08/918,401 filed Aug. 26, 1997, now abandoned, and of PCT/US97/15219 filed Aug. 27, 1997.

### BACKGROUND OF THE INVENTION

High level production of proteins in cell culture strongly depends on the ability to elicit specific and high-level expression of genes encoding RNAs or proteins of therapeutic, commercial, or experimental value. This problem is particularly acute when the gene to be expressed is an endogenous gene, i.e. one that is naturally present within a cell, because such genes are normally present in only a single copy. A variety of expression systems have been developed, including regulated expression systems, involving allosteric on switches triggered by tetracycline, RU486 and ecdysone, as well as dimerization based on-off switches triggered by FK1012, FK-CsA, rapamycin and analogs thereof. See e.g. Clackson, “Controlling mammalian gene expression with small molecules” Current Opinion in Chemical Biology 1997, 1:210-218. In each of these systems, various approaches for achieving high expression, including the search for stronger transcriptional promoters or higher transfection efficiencies, have in many cases not met with success. Meanwhile, in various lines of research with transcription factors, promising results in transient transfection models have not been borne out with chromosomally integrated reporter gene constructs. Furthermore, overexpression of transcription factors is commonly associated with toxicity to the host cell. We have developed, and describe herein, various improvements to transcription factors which facilitate high level, regulated expression of proteins in culture for protein production.

### SUMMARY OF THE INVENTION

The invention described herein provides methods for regulated production of a desired protein in cells. The method comprises providing cells containing recombinant nucleic acids encoding at least one fusion protein which binds to a selected ligand, wherein the fusion protein comprises a ligand binding domain and a DNA binding domain. In the presence of such a ligand, the cells express a gene operably linked to regulatory DNA to which said DNA binding domain binds. The cells are then exposed to a ligand under suitable conditions permitting gene expression and protein production by the cells in an amount sufficient for measurable expression of the gene and production of the encoded protein and the protein so produced is recovered from the cells. By recovery of the protein, we mean isolation of the desired protein from the culture medium, from cellular debris and/or from any unwanted cellular products which may be present. Any available methods and materials for recovery and purification of the desired protein may be adapted to the practice of this invention, including methods and materials which are well known, e.g. centrifugation and cellular fractionation; low and high pressure column chromatography (using affinity, ion exchange, gel filtration, reverse phase, hydrophobic interaction and/or hydroxyapatite); precipitation with high salt, low salt, organic solvents or polyethylene glycol; membrane filtration; detergent extraction; proteolytic digestion; preparative isoelectric focusing (IEF) or gel electrophoresis; and crystallization.

The selected cells may be any cells that can be expanded and maintained in culture, including mammalian cells, bacterial cells, insect cells and yeast cells. Preferably the cells are human cells and are used for production of a human protein. The cells should be maintained as a stable cell line, and the method of introduction of DNA into the cells can be any transduction method used in the art, including lipofection, calcium phosphate transfection, retroviral infection, electroporation, etc. The cell line used for protein production should preferably be certified to be free of mycoplasma, wild-type virus or other contaminants. The cells may be cultured by a variety of means including roller bottles, shaker flasks and fermenters. In most cases, the cells will be expanded in the absence of ligand and ligand added upon the cells reaching an appropriate density for protein production. The appropriate density for protein production will vary depending on the cell type. If desired, the cells may be continually maintained in the presence of ligand.

A wide variety of proteins may be targeted for production using these methods. For example, the protein produced may be a secreted protein such as erythropoietin, G-CSF, GM-CSF, leptin, an interleukin, VEGF, interferon alpha, beta or gamma, a neurotrophin, thrombopoietin, insulin, growth hormone, or a cytokine. Alternatively, the produced protein may be a cellular protein or membrane protein, which can be recovered from a cell lysate. A partial listing of target proteins which may be produced using the methods of this invention are listed in, e.g. U.S. Pat. No. 5,830,462.

The fusion proteins to be used in the methods of this invention may comprise additional domains, including bundling domains and transcription activation domains. The transcription activation domains can be composite activation domains, comprised of two or more activation units which are generally mutually heterologous. The activation domain may contain one or more synergizing domains to allow for increased expression. In a preferred embodiment, the DNA binding domain of the fusion protein binds a regulatory sequence of an endogenous gene. In some embodiments, the DNA binding domain is a composite DNA binding domain such as ZFHD1.

In a preferred embodiment, the method comprises providing cells containing recombinant nucleic acids encoding two fusion proteins which can bind simultaneously to a divalent (or multivalent) ligand, wherein: (i) the first fusion protein comprises a ligand binding domain and transcription activation domain, and (ii) the second fusion protein comprising a ligand binding domain and a DNA binding domain, and (iii) in the presence of such a ligand the cells express a gene operably linked to regulatory DNA to which said DNA binding domain binds; (b) exposing the cells to a divalent ligand in an amount sufficient for measurable expression of the gene and production of the encoded protein; and (c) recovering the protein so produced from the cells. In this embodiment, a preferred ligand binding domain is derived from an immunophilin domain such as FKBP12, and the fusion proteins are crosslinked upon binding to the ligand, as described in detail in U.S. Pat. No. 5,830,462, the full contents of which are incorporated herein by reference. Other regulated expression systems may also be used for production of proteins using the methods of this invention. For example, one allosterically regulated system uses a transcription factor containing a transcription activation domain, a DNA binding domain and ligand binding domain comprising a mutated progesterone receptor which binds to a progesterone analog. Binding of the ligand to the ligand binding domain of the transcription factor induces an allosteric change in the protein which allows the DNA binding domain to bind to an expression control sequence for the gene and activate transcription.

In an alternative embodiment, the fusion proteins comprise a conditional aggregation domain that causes the proteins to aggregate in the absence of ligand and be dispersed in the presence of ligand. In this embodiment, the method comprises (a) providing cells containing recombinant nucleic acids encoding two fusion proteins which self-aggregate in the absence of ligand, wherein: (i) the first fusion protein comprises a conditional aggregation domain which binds to a selected ligand and a transcription activation domain, and (ii)the second fusion protein comprising a conditional aggregation domain which binds to a selected and a DNA binding domain, and (iii)in the absence of ligand, the cells express a gene operably linked to regulatory DNA to which said DNA binding domain binds; (b) expanding the cells in the presence of ligand in an amount sufficient for repression of the gene; (c) removing the ligand to induce production of the encoded protein and (d) recovering the protein so produced from the cells. In some instances, the practitioner may desire to grow the cells entirely in the absence of ligand, thus allowing protein production to be continuous. Conditional aggregation domains and their uses are fully described in USSN 09/421,104, the full contents of which are incorporated herein by reference.

### BRIEF DESCRIPTION OF THE FIGURES

Abbreviations used in the Figures:

G=yeast GAL4 DNA binding domain, amino acids 1-94

F=human FKBP12, amino acids 1-107

R=FRB domain of human FRAP, amino acids 2025-2113

S=activation domain from the p65 subunit of human NF-kB, amino acids 361-550

V=activation domain from Herpesvirus VP16, amino acids 410-494

L=E. coli lactose repressor, amino acids 46-360

MT=Minimal Tetramerization (“bundling”) domain of E. coli lactose repressor, amino acids 324-360

FIG. 1 Diagram comparing various fusion proteins, with and without bundling domains, and their use in various strategies for delivery of activation domains to the promoter of a target gene.

FIG. 1A. Two fusion proteins, one containing a DNA binding domain (e.g. a GAL4 or ZFHD1 DNA binding domain) fused to an FKBP12, and the other containing a p65 activation domain fused to an FRB, are expressed in cells. Addition of rapamycin leads to the recruitment of a singe activation domain to each DNA binding domain monomer.

FIG. 1B. Fusion of multiple FKBPs to the DNA binding domain allows rapamycin to recruit multiple activation domains to each DNA binding domain monomer.

FIG. 1C. Addition of the lactose repressor tetramerization domain to the FRB-activation domain fusion allows rapamycin to recruit four activation domains to each FKBP fused to the DNA binding domain

FIG. 1D. Rapamycin recruits bundled activation domain fusion protein to each of the FKBP-DNA binding domain fusion proteins.

FIG. 1E. illustrates a mutated tetR-based system, without bundling.

FIG. 1F. illustrates a mutated tetR-based system, with bundling.

FIG. 1G. illustrates an engineered progesterone-R-based system, without bundling.

FIG. 1H. illustrates an engineered progesterone-R-based system, with bundling.

FIG. 2 Expression levels of the stably integrated reporter gene correlate with the number of activation domains recruited to the promoter. The indicated DNA binding domain and activation domain fusions were transfected into HT1080B cells containing a stably integrated SEAP reporter. Mean values of SEAP activity secreted into the medium following addition of 10 nM rapamycin are shown (+/−S.D.). In all cases, SEAP expression values are plotted for cultures receiving 100 ng of activation domain expression plasmid, which gives peak expression values in transiently transfected cells and slightly below peak levels in the stably transfected cell line.

FIG. 3 A thirty-six amino acid region in the carboxy terminal of the lactose repressor protein is sufficient for generating highly potent and bundled activation domain fusion proteins. HT1080 B cells were co-transfected with 20 ng GF1 and 100 ng of indicated activation domain containing plasmid vectors. Transcription of the reporter gene was stimulated by the addition of 10 nM rapamycin in the medium. Mean values of SEAP activity secreted into the medium assayed 24 hrs after transfection are shown (+/−S.D.)

FIG. 4A Tethering bundled activation domain fusion proteins to DNA binding proteins significantly reduces the amount of reconstituted activators required to strongly stimulate the target gene expression. Twenty nanograms of GF4 and indicated concentrations of activation domain expressing plasmids were transfected into HT1080 B cells. Transcription of the stably integrated reporter gene was induced by the addition of 10 nM rapamycin in the medium.

FIG. 4B Western blot analysis of the relative expression levels of the transfected transcription factors.

FIG. 4C Twenty nanograms of GF4 and one hundred nanograms of the indicated activation domain fusion protein encoding plasmids were co-transfected into HT1080 B cells and the transcriptional activity of the GAL4 responsive reporter gene was induced by the addition of indicated concentrations of rapamycin in the medium. In all cases, mean values of SEAP activity secreted into the medium 24 hrs after the addition of rapamycin are shown (+/−S.D.).

### DETAILED DESCRIPTION OF THE INVENTION

Definitions

For convenience, the intended meaning of certain terms and phrases used herein are provided below.

“Activate” as applied to the expression or transcription of a gene denotes a directly or indirectly observable increase in the production of a gene product, e.g., an RNA or polypeptide encoded by the gene.

“Conditional aggregation domains” (CADs) are domains which form aggregates with one another which are dispersed in the presence of ligand. Fusion proteins containing CADs are retained in cellular compartments, e.g. the cytoplasm or the nucleus. Such fusion proteins can also have nuclear localization sequences, which target the aggregates to the nucleus.

In a preferred embodiment, the CAD is derived from human FKBP12. In particular, the FKBP mutant F36M functions as a conditional aggregation domain when fused to a heterologous target sequence in eukaryotic, e.g. mammalian, cells. In the absence of ligand, fusion proteins containing FKBP F36M self-aggregate and accumulate in complexes. Upon addition of ligand, the fusion protein disaggregates. Another FKBP mutant which functions as a CAD is FKBP W59V.

“Capable of selectively hybridizing” means that two DNA molecules are susceptible to hybridization with one another, despite the presence of other DNA molecules, under hybridization conditions which can be chosen or readily determined empirically by the practitioner of ordinary skill in this art. Such treatments include conditions of high stringency such as washing extensively with buffers containing 0.2 to 6× SSC, and/or containing 0.1% to 1% SDS, at temperatures ranging from room temperature to 65-75° C. See for example F. M. Ausubel et al., Eds, Short Protocols in Molecular Biology, Units 6.3 and 6.4 (John Wiley and Sons, New York, 3d Edition, 1995).

“Cells”, “host cells” or “recombinant host cells” refer not only to the particular cells under discussion, but also to their progeny or potential progeny. Because certain modifications may occur in succeeding generations due to either mutation or environmental influences, such progeny may not, in fact, be identical to the parent cell, but are still included within the scope of the term as used herein.

“Cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

“Composite”, “fusion”, and “recombinant” denote a material such as a nucleic acid, nucleic acid sequence or polypeptide which contains at least two constituent portions which are mutually heterologous in the sense that they are not otherwise found directly (covalently) linked in nature, i.e., are not found in the same continuous polypeptide or gene in nature, at least not in the same order or orientation or with the same spacing present in the composite, fusion or recombinant product. Typically, such materials contain components derived from at least two different proteins or genes or from at least two non-adjacent portions of the same protein or gene. In general, “composite” refers to portions of different proteins or nucleic acids which are joined together to form a single functional unit, while “fusion” generally refers to two or more functional units which are linked together. “Recombinant” is generally used in the context of nucleic acids or nucleic acid sequences.

“Cofactor” refers to proteins which either enhance or repress transcription in a non-gene specific manner. Cofactors typically lack intrinsic DNA binding specificity, and function as general effectors. Positively acting cofactors do not stimulate basal transcription, but enhance the response to an activator. Positively acting cofactors include PC1, PC2, PC3, PC4, and ACF. TAFs which interact directly with transcriptional activators are also referred to as cofactors.

A “coding sequence” or a sequence which “encodes” a particular polypeptide or RNA, is a nucleic acid sequence which is transcribed (in the case of DNA) and translated (in the case of mRNA) into a polypeptide in vitro or in vivo when placed under the control of an appropriate expression control sequence. The boundaries of the coding sequence are generally determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and synthetic DNA sequences. A transcription termination sequence will usually be located 3′ to the coding sequence.

A “construct”, e.g., a “nucleic acid construct” or “DNA construct”, refers to a nucleic acid or nucleic acid sequence.

“Derived from” denotes a peptide or nucleotide sequence selected from within a given sequence. A peptide or nucleotide sequence derived from a named sequence may further contain a small number of modifications relative to the parent sequence, in most cases representing deletion, replacement or insertion of less than about 15%, preferably less than about 10%, and in many cases less than about 5%, of amino acid residues or bases present in the parent sequence. In the case of DNAs, one DNA molecule is also considered to be derived from another if the two are capable of selectively hybridizing to one another. Polypeptides or polypeptide sequences are also considered to be derived from a reference polypeptide or polypeptide sequence if any DNAs encoding the two polypeptides or sequences are capable of selectively hybridizing to one another. Typically, a derived peptide sequence will differ from a parent sequence by the replacement of up to 5 amino acids, in many cases up to 3 amino acids, and very often by 0 or 1 amino acids. A derived nucleic acid sequence will differ from a parent sequence by the replacement of up to 15 bases, in many cases up to 9 bases, and very often by 0-3 bases. In some cases the amino acid(s) or base(s) is/are added or deleted rather than replaced.

“Domain” refers to a portion of a protein or polypeptide. In the art, the term “domain” may refer to a portion of a protein having a discrete secondary structure. However, as will be apparent from the context used herein, the term “domain” as used in this document does not necessarily connote a given secondary structure. Rather, a peptide sequence is referred to herein as a “domain” simply to denote a polypeptide sequence from a defined source, or having or conferring an intended or observed activity. Domains can be derived from naturally occurring proteins or may comprise non-naturally-occurring sequence.

“DNA recognition sequence” means a DNA sequence which is capable of binding to one or more DNA-binding domains, e.g., of a transcription factor or an engineered polypeptide.

“Expression control element”, or simply “control element”, refers to DNA sequences, such as initiation signals, enhancers, promoters and silencers, which induce or control transcription of DNA sequences with which they are operably linked. Control elements of a gene may be located in introns, exons, coding regions, and 3′ flanking sequences. Some control elements are “tissue specific”, i.e., affect expression of the selected DNA sequence preferentially in specific cells (e.g., cells of a specific tissue), while others are active in many or most cell types. Gene expression occurs preferentially in a specific cell if expression in this cell type is observably higher than expression in other cell types. Control elements include so-called “leaky” promoters, which regulate expression of a selected DNA primarily in one tissue, but cause expression in other tissues as well. Furthermore, a control element can act constitutively or inducibly. An inducible promoter, for example, is demonstrably more active in response to a stimulus than in the absence of that stimulus. A stimulus can comprise a hormone, cytokine, heavy metal, phorbol ester, cyclic AMP (cAMP), retinoic acid or derivative thereof, etc. A nucleotide sequence containing one or more expression control elements may be referred to as an “expression control sequence”.

“Gene” refers to a nucleic acid molecule or sequence comprising an open reading frame and including at least one exon and (optionally) one or more intron sequences.

“Genetically engineered cells” denotes cells which have been modified by the introduction of recombinant or heterologous nucleic acids (e.g. one or more DNA constructs or their RNA counterparts) and further includes the progeny of such cells which retain part or all of such genetic modification.

“Heterologous”, as it relates to nucleic acid or peptide sequences, denotes sequences that are not normally joined together, and/or are not normally associated with a particular cell. Thus, a “heterologous” region of a nucleic acid construct is a segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, in the case of a cell transduced with a nucleic acid construct which is not normally present in the cell, the cell and the construct would be considered mutually heterologous for purposes of this invention. Allelic variation or naturally occurring mutational events do not give rise to heterologous DNA, as used herein.

“Interact” refers to directly or indirectly detectable interactions between molecules, such as can be detected using, for example, a yeast two hybrid assay or by immunoprecipitation. The term “interact” encompasses “binding” interactions between molecules. Interactions may be, for example, protein-protein, protein-nucleic acid, protein-small molecule or small molecule-nucleic acid in nature.

“Minimal promoter” refers to the minimal expression control element that is capable of initiating transcription of a selected DNA sequence to which it is operably linked. A minimal promoter frequently consists of a TATA box or TATA-like box. Numerous minimal promoter sequences are known in the literature.

“Nucleic acid” refers to polynucleotides such as deoxyribonucleic acid (DNA), and, where appropriate, ribonucleic acid (RNA). The term should also be understood to include derivatives, variants and analogs of either RNA or DNA made from nucleotide analogs, and, as applicable to the embodiment being described, single (sense or antisense) and double-stranded polynucleotides.

“Operably linked” when referring to an expression control element and a coding sequence means that the expression control element is associated with the coding sequence in such a manner as to permit or facilitate transcription of the coding sequence.

“Protein”, “polypeptide” and “peptide” are used interchangeably.

A “target gene” is a nucleic acid of interest, generally endogenous to the cell, the expression of which is modulated according to the methods of the invention.

The terms “transcriptional activation unit” and “activation unit”, refer to a peptide sequence which is capable of inducing or otherwise potentiating transcription activator-dependent transcription, either on its own or when linked covalently or non-covalently to another transcriptional activation unit. An activation unit may contain a minimal polypeptide sequence which retains the ability to interact directly or indirectly with a transcription factor. Unless otherwise clear from the context, where a fusion protein is referred to as “including” or “comprising” an activation unit, it will be understood that other portions of the protein from which the activation unit is derived can be included. Transcriptional activation units can be rich in certain amino acids. For example, a transcriptional activation unit can be a peptide rich in acidic residues, glutamine, proline, or serine and threonine residues. Other transcriptional activators can be rich in isoleucine or basic amino acid residues (see, e.g., Triezenberg (1995) Cur. Opin. Gen. Develop. 5:190, and references cited therein). For instance, an activation unit can be a peptide motif of at least about 6 amino acid residues associated with a transcription activation domain, including the well-known “acidic”, “glutamine-rich” and “proline-rich” motifs such as the K13 motif from p65, the OCT2 Q domain and the OCT2 P domain, respectively.

The term “transcriptional activator” refers to a protein or protein complex, the presence of which can increase the level of gene transcription in a cell of a responsive gene. It is thought that a transcriptional activator is capable of enhancing the efficiency with which the basal transcription complex performs, i.e., activating transcription. Thus, as used herein, a transcriptional activator can be a single protein or alternatively it can be composed of several units at least some of which are not covalently linked to each other. A transcriptional activator typically has a modular structure, i.e., comprises one or more component domains, such as a DNA binding domain and one or more transcriptional activation units or domains. Transcriptional activators are a subset of transcription factors, defined below.

“Transcription factor” refers to any protein whose presence or absence contributes to the initiation of transcription but which is not itself a part of the polymerase. Certain transcription factors stimulate transcription (“transcriptional activators”); other repress transcription (“transcriptional repressors”). Transcription factors are generally classifiable into two groups: (i) the general transcription factors, and (ii) the transcription activators. Transcription factors usually contain one or more regulatory domains. Some transcription factors contain a DNA binding domain, which is that part of the transcription factor which directly interacts with the expression control element of the target gene.

“Transcription regulatory domain” denotes any domain which regulates transcription, and includes activation, synergizing and repression domains. The term “activation domain” denotes a domain, e.g. in a transcription factor, which positively regulates (increases) the rate of gene transcription. The term “repression domain” denotes a domain which negatively regulates (inhibits or decreases) the rate of gene transcription.

A “transcription synergizing domain” is defined as any domain which increases the potency of transcriptional activation when present along with the transcription activation domain. A synergizing domain can be an independent transcriptional activator, or alternatively, a domain which on its own does not induce (or does not usually induce) transcription but is able to potentiate the activity of a transcription activation domain. The synergizing domain can be a component domain of a fusion protein containing the activation domain or can be recruited to the DNA binding domain or other component of the transcription complex, e.g., via a bundling interaction.

“Transfection” means the introduction of a naked nucleic acid molecule into a recipient cell. “Infection” refers to the process wherein a nucleic acid is introduced into a cell by a virus containing that nucleic acid. A “productive infection” refers to the process wherein a virus enters the cell, is replicated, and is then released from the cell (sometimes referred to as a “lytic” infection). “Transduction” encompasses the introduction of nucleic acid into cells by any means.

“Transgene” refers to a nucleic acid sequence which has been introduced into a cell. Daughter cells deriving from a cell in which a transgene has been introduced are also said to contain the transgene (unless it has been deleted). The polypeptide or RNA encoded by a transgene may be partly or entirely heterologous, i.e., foreign, with respect to the animal or cell into which it is introduced. Alternatively, the transgene can be homologous to an endogenous gene of the transgenic animal or cell into which it is introduced, but is designed to be inserted, or is inserted, into the animal's genome in such a way as to alter the genome of the cell into which it is inserted (e.g., it is inserted at a location which differs from that of the natural gene). A transgene can also be present in an episome. A transgene can include one or more expression control elements and any other nucleic acid, (e.g. intron), that may be necessary or desirable for optimal expression of a selected coding sequence.

The term “vector” refers to a nucleic acid molecule capable of transporting another nucleic acid to which it has been linked. One type of vector is an episome, i.e., a nucleic acid capable of extra-chromosomal replication. Often vectors are used which are capable of autonomous replication and/or expression of nucleic acids to which they are linked. Vectors capable of directing the expression of an included gene operatively linked to an expression control sequence can be referred to as “expression vectors”. Expression vectors are typically in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of vectors which serve equivalent functions and which are or become known in the art. Viral vectors are nucleic acid molecules containing viral sequences which can be packaged into viral particles.

Bundling Domains

As described above, bundling domains interact with like domains via protein-protein interactions to induce formation of protein “bundles”. Various order oligomers (dimers, trimers, tertramers, etc.) of proteins containing a bundling domain can be formed, depending on the choice of bundling domain.

One example of a dimerization domain is the leucine zipper (LZ) element. Leucine zippers have been identified, generally, as stretches of about 35 amino acids containing 4-5 leucine residues separated from each other by six amino acids (Maniatis and Abel (1989) Nature 341 :24-25). Exemplary leucine zippers occur in a variety of eukaryotic DNA binding proteins, such as GCN4, C/EBP, c-Fos, c-Jun, c-Myc and c-Max. Other dimerization domains include helix-loop-helix domains (Murre, C. et al. (1989) Cell 58:537-544). Dimerization domains may also be selected from other proteins, such as the retinoic acid receptor, the thyroid hormone receptor or other nuclear hormone receptors (Kurokawa et al. (1993) Genes Dev. 7:1423-1435) or from the yeast transcription factors GAL4 and HAP1 (Marmonstein et al. (1992) Nature 356:408-414; Zhang et al. (1993) Proc. Natl. Acad. Sci. USA 90:2851-2855). Dimerization domains are further described in U.S. Pat. No. 5,624,818 by Eisenman.

Of particular current interest are tetramer-forming bundling domains. Incorporation of such a tetramerization domain within a fusion protein leads to the constitutive assembly of tetrameric clusters or bundles. For example, a bundle of four activation units can be assembled by covalently linking the activation unit to a tetramerization domain. By clustering the activation units together through a bundling domain, four activation units can be delivered to a single DNA binding domain at the promoter. The E. coli lactose repressor tetramerization domain (amino acids 46-360; Chakerian et al. (1991) J. Biol. Chem. 266:1371; Alberti et al. (1993) EMBO J. 12:3227; and Lewis et al. (1996) Nature 271:1247), illustrates this class. Furthermore, since the fusion proteins may contain more than one activation unit linked to the bundling domain, each of the four proteins of the tetramer can contain more than one activation unit (and the complex may comprise more than 4 activation units).

Other illustrative tetramerization domains include those derived from residues 322-355 of p53 (Wang et al. (1994) Mol. Cell. Biol. 14:5182; Clore et al. (1994) Science 265:386) see also U.S. Pat. No. 5,573,925 by Halazonetis. Other bundling domains can be derived from the Dimerization cofactor of hepatocyte nuclear factor-1 (DCoH). DCoH associates with specific DNA binding proteins and also catalyses the dehydration of the biopterin cofactor of phenylalanine hydroxylase. DCoH is a tetramer. See e.g. Endrizzi, J. A., Cronk, J. D., Wang, W., Crabtree, G. R and Alber, T. (1995) Science 268, 556559; Suck and Ficner (1996) FEBS Lett 389(1):3-39; Standmann, Senkel and Ryffel (1998) Int J Dev Biol 42(1):53-59.

Other bundling domains may be trimerization domains, for example, the trimerization domains of human heat shock factor 1, TRAF-2, lung surfactant protein D or clathrin.

The bundling domain may comprise a naturally-occurring peptide sequence or a modified or artificial peptide sequence. Sequence modifications in the bundling domain may be used to increase the stability of bundle formation or to help avoid unintended bundling with native protein molecules in the engineered cells which contain a wild-type bundling domain.

For example, sequence substitutions that stabilize oligomerization driven by leucine zippers are known (Krylov et al. (1994) cited above; O'Shea et al. (1992) cited above). To illustrate, residues 174 or 175 of human p53 may be replaced by glutamine or leucine, respectively.

To illustrate sequence modifications aimed at avoiding unintended bundling with endogenous protein molecules, the p53 tetramerization domain may be modified to reduce the likelihood of bundling with endogenous p53 proteins that have a wild-type p53 tetramerization domain, such as wild-type p53 or tumor-derived p53 mutants. Such altered p53 tetramerization domains are described in U.S. Pat. No. 5,573,925 by Halazonetis and are characterized by disruption of the native p53 tetramerization domain and insertion of a heterologous bundling domain in a way that preserves tetramerization. Disruption of the p53 tetramerization domain involving residues 335-348, or a subset of these residues, sufficiently disrupts the function of this domain so that it can no longer drive tetramerization with wild-type p53 or tumor-derived p53 mutants. At the same time, however, introduction of a heterologous dimerization domain reestablishes the ability to form tetramers, which is mediated both by the heterologous dimerization domain and by the residual portion of the p53 tetramerization domain sequence.

Other suitable bundling domains can be readily selected or designed by the practitioner, including semi-artificial bundling domains, such as variants of the GCN4 leucine zipper that form tetramers (Alberti et al. (1993) EMBO J. 12:3227-3236; Harbury et al. (1993) Science 262:1401-1407; Krylov et al. (1994) (1994) EMBO J. 13:2849-2861). The tetrameric variant of GCN4 leucine zipper described in Harbury et al. (1993), supra, has isoleucines at positions d of the coiled coil and leucines at positions a, in contrast to the original zipper which has leucines and valines, respectively.

The choice of bundling domain can be based, at least in part, on the desired conformation of the bundles. For instance, the GCN4 leucine zipper drives parallel subunit assembly [Harbury et al. (1993), cited above], while the native p53 tetramerization domain drives antiparallel assembly [Clore et al. (1994) cited above; Sakamoto et al. (1994) Proc. Natl. Acad. Sci. USA 91:8974-8978].

In addition, a variety of techniques are available for identifying other naturally occurring bundling domains, as well as for selecting bundling domains derived from mutant or otherwise artificial sequences. See, for example, Zeng et al. (1997) Gene 185:245; O'Shea et al. (1992) Cell 68:699-708; Krylov et al. [cited above].

In some cases, the use of bundling domains of the same species as the desired cell line may induce interactions between the fusion proteins and the endogenous protein from which the bundling domain was derived, i.e., leading to unwanted bundling of fusion proteins with the endogenous protein containing the identical bundling domain. Such interactions, in addition to inhibiting target gene expression, may also have other adverse effects in the cell, e.g., by interfering with the function of the endogenous protein from which the bundling domain was derived.

Approaches for avoiding unwanted bundling of fusion proteins of this invention with endogenous proteins include using a bundling domain which is (a) heterologous to the host organism, (b) expressed by the host organism but only (or predominantly) in cells or tissues other than those which will express the fusion proteins, or (c) engineered through modification in peptide sequence such that it bundles preferentially with itself rather than with an endogenous bundling domain.

The first approach is illustrated by the use of a bacterial lac repressor tetramerization domain in human cells.

The second approach requires the use of a bundling domain derived from a protein which is not expressed in the cells which are to be engineered to express the fusion protein(s) of this invention, at least not at a level which would cause undue interference with the bundling application or with normal cell function. Fusion proteins containing a bundling domain derived from an endogenous protein expressed selectively or preferentially in one tissue could be expressed in cells derived from a different tissue without any adverse effects. For example, to regulate gene expression in human muscle cells, fusion proteins containing bundling domains from a protein expressed in liver, brain or some other tissue or tissues—but not in muscle—can be expressed in muscle cells without undue risk of mismatched bundling.

In the third approach, and as noted previously, the binding specificity of the bundling domain is engineered by alterations in peptide sequence to replace (in whole or part) bundling activity for proteins containing the wild-type bundling domain with bundling activity for proteins containing the modified peptide sequence.

Several examples of tissue-specific bundling domains which could be used in the practice of this invention include bundling domains derived from the Retinoid X receptor, (Kersten, S., Reczek, P. R and N. Noy (1997) J. Biol. Chem. 272, 29759-29768); Dopamine D3 receptor (Nimchinsky, E. A., Hof, P. R., Janssen, W. G. M., Morrison, J. H and C. Schmauss (1997) J. Biol. Chem. 272, 29229-29237); Butyrylcholinesterase (Blong, R.M., Bedows, E and O. Lockridge (1997) Biochem. J. 327, 747-757); Tyrosine Hydroxylase (Goodwill, K. E., Sabatier, C., Marks, C., Raag, R., Fitzpatrick, P. F and R. C. Stevens (1997) Nat. Struct. Biol 7, 578-585). Bcr (McWhirter, J. R., Galasso, D. L and J. Y. Wang (1993) Mol. Cell. Biol. 13, 7587-7595); and Apolipoprotein E (Westerlund, J. A and K. H. Weisgraber (1993) J. Biol. Chem. 268, 15745-15750).

Transcription Activation Domains/Activation Units

Transcription activation domains and activation units can comprise naturally-occurring or non-naturally-occurring peptide sequence so long as they are capable of activating or potentiating transcription of a target gene construct. A variety of polypeptides and polypeptide sequences which can activate or potentiate transcription in eukaryotic cells are known and in many cases have been shown to retain their activation function when expressed as a component of a fusion protein. An activation unit is generally at least 6 amino acids, and preferably contains no more than about 300 amino acid residues, more preferably less than 200, or even less than 100 residues.

Naturally occurring activation units include portions of transcription factors, such as a thirty amino acid sequence from the C-terminus of VP16 (amino acids 461-490), referred to herein as “Vc”. Other activation units are derived from naturally occurring peptides. For example, the replacement of one amino acid of a naturally occurring activation unit by another may further increase activation. An example of such an activation unit is a derivative of an eight amino acid peptide of VP16, the derivative having the amino acid sequence DFDLDMLG. Other activation units are “synthetic” or “artificial” in that they are not derived from a naturally occurring sequence. It is known, for example, that certain random alignments of acidic amino acids are capable of activating transcription.

Certain transcription factors are known to be active only in specific cell types, i.e., they activate transcription in a tissue specific manner. By using activation units which function selectively or preferentially in specific cells, it is possible to design a transcriptional activator of the invention having a desired tissue specificity.

One source of peptide sequence for use in a fusion protein of this invention is the herpes simplex virus virion protein 16 (referred to herein as VP16, the amino acid sequence of which is disclosed in Triezenberg, S.J. et al. (1988) Genes Dev. 2:718-729). For example, an activation unit corresponding to about 127 of the C-terminal amino acids of VP16 can be used. Alternatively, at least one copy of about 11 amino acids from the C-terminal region of VP16 which retains transcription activation ability is used as an activation unit. Preferably, an oligomer comprising two or more copies of this sequence is used. Suitable C-terminal peptide portions of VP16 include those described in Seipel, K. et al. (EMBO J. (1992) 13:4961-4968).

Another example of an acidic activation unit is provided in residues 753-881 of GAL4.

One particularly important source of transcription activation units is the (human) NF-kB subunit p65. The activation domain may contain one or more copies of a peptide sequence comprising all or part of the p65 sequence spanning residues 450-550, or a peptide sequence derived therefrom. In certain embodiments, it has been found that extending the p65 peptide sequence to include sequence spanning p65 residues 361-450, e.g., including the “AP activation unit”, leads to an unexpected increase in transcription activation. Moreover, a peptide sequence comprising all or a portion of p65(361-550), or peptide sequence derived therefrom, in combination with heterologous activation units, can yield surprising additional increases in the level of transcription activation. p65-based activation domains function across a broad range of promoters and in a number of bundling experiments have yielded increases in transcription levels of chromosomally incorporated target genes six-fold, eight-fold and even 14-15-fold higher than obtained with unbundled tandem copies of VP16 which itself is widely recognized as a very potent activation domain.

It is expected that recombinant DNA molecules encoding fusion proteins which contain a p65 activation unit, or peptide sequence derived therefrom, will provide significant advantages for heterologous gene expression in its various contexts, including dimerization based regulated systems such as described in International patent applications PCT/US94/01617, PCT/US95/10591, PCT/US96/09948 and the like, as well as in other heterologous transcription systems including allostery-based regulation such as those involving tetracycline-based regulation reported by Bujard et al. and those involving steroid or other hormone-based regulation.

One class of p65-based transcription factors contain more than one copy of a p65-derived domain. Such proteins will typically contain two or more, generally up to about six, copies of a peptide sequence comprising all or a portion of p65(361-550), or peptide sequence derived therefrom. Such iterated p65-based transcription activation domains are useful both in bundled and non-bundled approaches.

Other polypeptides with transcription activation activity in eukaryotic cells can be used to provide activation units for the fusion proteins of this invention. Transcription activation domains found within various proteins have been grouped into categories based upon shared structural features. Types of transcription activation domains include acidic transcription activation domains (noted previously), proline-rich transcription activation domains, serine/threonine-rich transcription activation domains and glutamine-rich transcription activation domains. Examples of proline-rich activation domains include amino acid residues 399-499 of CTF/NF1 and amino acid residues 31-76 of AP2. Examples of serine/threonine-rich transcription activation domains include amino acid residues 1-427 of ITF1 and amino acid residues 2-451 of ITF2. Examples of glutamine-rich activation domains include amino acid residues 175-269 of Oct1 and amino acid residues 132-243 of Sp1. The amino acid sequences of each of the above described regions, and of other useful transcription activation domains, are disclosed in Seipel, K. et al. (EMBO J. (1992) 13:4961-4968).

Still other illustrative activation domains and motifs of human origin include the activation domain of human CTF, the 18 amino acid (NFLQLPQQTQGALLTSQP) glutamine rich region of Oct-2, the N-terminal 72 amino acids of p53, the SYGQQS repeat in Ewing sarcoma gene and an 11 amino acid (535-545) acidic rich region of Rel A protein.

In addition to previously described transcription activation domains, novel transcription activation units, which can be identified by standard techniques, are within the scope of the invention. The transcription activation ability of a polypeptide can be assayed by linking the polypeptide to a DNA binding domain and determining the amount of transcription of a target sequence that is stimulated by the fusion protein. For example, a standard assay used in the art utilizes a fusion protein of a putative activation unit and a GAL4 DNA binding domain (e.g., amino acid residues 1-93). This fusion protein is then used to stimulate expression of a reporter gene linked to GAL4 binding sites (see e.g., Seipel, K. et al. (1992) EMBO J. 11:4961-4968 and references cited therein).

The activation domains of the invention can be from any eukaryotic species (including but not limited to various yeast species and various vertebrate species, including the mammals), and it is not necessary that every activation unit or domain be from the same species. In applications of this invention to whole organisms, it is often preferable to use activation units and activation domains from the same species as the recipient to avoid immune reactions against the fusion proteins.

Techniques for making the subject fusion proteins are adapted from well-known procedures. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments. Amplification products can subsequently be annealed to generate a chimeric gene sequence (see, for example, Current Protocols in Molecular Biology, Eds. Ausubel et al. John Wiley & Sons: 1992).

Synergizing Domains

A synergizing domain is any domain which observably increases the potency of transcription activation when recruited to the promoter along with the transcription activation domain. A synergizing domain can be an independent transcription activation domain or an activation unit which on its own does not induce transcription but is able to potentiate the activity of a transcription activation domain with which it is linked covalently (i.e., within the same fusion protein) or with which it is associated non-covalently (e.g., through bundling or ligand-mediated clustering).

One example of a synergizing domain is the so-called “alanine/proline rich” or “AP” activation motif of p65, which extends from about amino acids 361 to about amino acid 450 of that protein. Similar AP activation motifs are also present in, e.g., the p53 and CTF proteins. The presence of one or several copies of an AP domain alone in a protein does not itself provide the ability to induce activator-dependent transcription activation. However, when linked to activation units which are themselves capable of inducing some level of activator-dependent transcription, e.g., another portion of p65 or VP16, the AP activation unit synergizes with the second activation domain to induce an increase in the level of transcription.

Accordingly, the invention provides an AP activation unit, functional derivative thereof, or other synergizing domain which on its own is incapable of activating transcription. Functional alternative sequences for use as synergizing domains, including among others derivatives of an AP activation unit, can be obtained, for instance, by screening candidate sequences for binding to TFIIA and measuring transcriptional activity in a co-transfection assay. Such equivalents are expected to include forms of the activation unit which are truncated at either the N-terminus or C-terminus or both, e.g., fragments of p65 (or homologous sequences thereto) which are about 75, 60, 50, 30 or even 20 amino acid residues in length (e.g., ranging in length from 20-89 amino acids). Likewise, it is expected that the AP activation unit sequence from p65 can tolerate amino acid substitutions, e.g., to produce AP motifs of at least 95%, 90%, 80% and even 70% identity with the AP activation unit sequence of SEQ ID No. 2 of U.S. Ser. No. 08/918,401. These and other AP derivatives include, for example, AP domains based on naturally-occurring sequence but modified by the replacement, insertion or deletion of 1, 2, 3, 4 or 5 amino acid residues.

Other synergizing domains are independent activation domains, e.g. VP16. While VP16 can activate transcription on its own, it can synergize with p65 to produce levels of transcription that are greater than the sum of the transcription levels effected by each activation domain alone. As shown in the examples, fusion of VP16 to a nucleic acid containing an FRB domain, a lac repressor tetramerization domain and p65 greatly increases the level of expression of a target gene as compared to the same construct in the absence of VP16.

Synergizing domains may also be fused to an unbundled or bundled DNA binding domain. To avoid the activation of transcription in a constitutive manner with constructs such as these, it is preferable that the synergizing domain itself be incapable of activating transcription.

DNA Binding Domains

Regulated expression systems relevant to this invention involve the use of a protein containing a DNA binding domain to selectively target a desired endogenous gene for expression (or repression). Systems based on ligand-mediated cross-linking generally rely upon a fusion protein containing the DNA binding domain together with one or more ligand binding domains. One general advantage of such systems is that they are particularly modular in nature and lend themselves to a wide variety of design choices. These systems permit wide latitude in the choice of DNA binding domains and allow the practitioner to select a DNA binding domain that interacts with the promoter of the endogenous gene to be expressed. Of the allostery-based systems, the progesterone receptor-based system and like systems permit relatively greater latitude in the choice of DNA binding domain than systems like those regulated by tetracycline or ecdysone.

Various DNA binding domains may be incorporated into the design of fusion proteins of this invention, especially those of the ligand-mediated cross-linking type and the progesterone-R-based type, so long as a corresponding DNA “recognition” sequence is known, or can be identified, to which the domain is capable of binding. One or more copies of the recognition sequence are incorporated into, or present within, the expression control sequence of the target gene construct.

The DNA binding domain can be a naturally occurring DNA-binding domain from a transcription factor. Alternatively, the DNA binding domain can be an artificial (or partially artificial) polypeptide sequence having DNA binding activity. For example, the DNA-binding domain can be a naturally occurring DNA binding domain that has been modified to recognize a different DNA binding site. The particular DNA-binding domain chosen will depend on the target promoter. For example, if the gene to be transcriptionally activated by the subject method is an endogenous gene, the DNA-binding domain must be able to interact with the promoter of the endogenous gene (endogenous promoter). Alternatively, as described in greater detail below, the endogenous promoter could be replaced, e.g., by homologous recombination, with a heterologous promoter for which the DNA binding domain is selected. Such a substitution may be necessary if no transcription factor is known to bind the endogenous promoter of interest. Alternatively, in such a situation, it is also possible to clone a DNA-binding domain interacting specifically with a sequence in the promoter of interest. This can be done, e.g., by phage display screening with a DNA molecule comprising at least a portion of the promoter of interest.

Desirable properties of DNA binding domains include high affinity for specific nucleotide sequences, termed herein “target sequences”, low affinity for most other sequences in a complex genome (such as a mammalian genome), low dissociation rates from specific DNA sites, and novel DNA recognition specificities distinct from those of known natural DNA-binding proteins. Preferably, binding of a DNA-binding domain to a specific target sequence is at least two, more preferably three and even more preferably more than four orders of magnitude greater than binding to any one alternative DNA sequence, as may be measured by relative Kd values or by relative rates or levels of transcription of genes associated with the selected and any alternative DNA sequences. It is also preferred that the selected DNA sequence be recognized to a substantially greater degree by the DNA binding domain of the transcriptional activator of the invention than by an endogenous DNA binding protein. Thus, for example, target gene expression in a cell is preferably two, more preferably three, and even more preferably more than four orders of magnitude greater in the presence of the transcriptional activator of the invention containing a DNA-binding region than in its absence.

Preferred DNA binding domains have a dissociation constant for a target sequence below 10⁻⁸ M, preferably 10⁻⁹ M, more preferably below 10⁻¹⁰ M, even more preferably below 10⁻¹¹ M.

From a structural perspective, DNA-binding that can be used in the invention may be classified as DNA-binding proteins with a helix-turn-helix structural design, such as, but not limited to, Myb, Ultrabithorax, Engrailed, Paired, Fushi tarazu, HOX, Unc86, the Ets and homeobox families of transcription factors, and the previously noted Oct1, Oct2 and Pit; zinc finger proteins, such as Zif268, SWI5, Kr,ppel and Hunchback; steroid receptors; DNA-binding proteins with the helix-loop-helix structural design, such as Daughterless, Achaete-scute (T3), MyoD, E12 and E47; and other helical motifs like the leucine-zipper, which includes GCN4, C/EBP, c-Fos/c-Jun and JunB. The amino acid sequences of the component DNA-binding domains may be naturally-occurring or non-naturally-occurring (or modified). DNA-binding domains and their target sites can be found at TF SEARCH (http://www.genome.ad:jp/SIT/TFSEARCH html). Another publicly available database of transcription factors and the sequences to which they bind is available from the National Library of Medicine in the “Transcription Data Base”.

One strategy for obtaining component DNA-binding domains with properties suitable for this invention is to modify an existing DNA-binding domain to reduce its affinity for DNA into the appropriate range. For example, a homeodomain such as that derived from the human transcription factor Phox1, may be modified by substitution of the glutamine residue at position 50 of the homeodomain. Substitutions at this position remove or change an important point of contact between the protein and one or two base pairs of the 6-bp DNA sequence recognized by the protein. Thus, such substitutions reduce the free energy of binding and the affinity of the interaction with this sequence and may or may not simultaneously increase the affinity for other sequences. Such a reduction in affinity is sufficient to effectively eliminate occupancy of the natural target site by this protein when produced at typical levels in mammalian cells. But it would allow this domain to contribute binding energy to and therefore cooperate with a second linked DNA-binding domain. Other domains that amenable to this type of manipulation include the paired box, the zinc-finger class represented by steroid hormone receptors, the myb domain, and the ets domain.

In another embodiment, the DNA binding domain is created from the assembly of DNA binding domains from various transcription factors, resulting in a DNA binding domain having a novel DNA binding specificity. Such composite DNA binding domains provide one means for achieving novel sequence specificity for the protein-DNA binding interaction. An illustrative composite DNA binding domain containing component peptide sequences of human origin is ZFHD-1 which is comprised of an Oct-1 homeodomain and zinc fingers 1 and 2 of Zif268, and is further described in PCT Application WO 96/20951 by Pomerantz et al. Individual DNA-binding domains may be further modified by mutagenesis to decrease, increase, or change the recognition specificity of DNA binding. These modifications can be achieved by rational design of substitutions in positions known to contribute to DNA recognition (often based on homology to related proteins for which explicit structural data are available).

The DNA sequences recognized by a chimeric protein containing a composite DNA-binding domain can be determined experimentally, as described below, or the proteins can be manipulated to direct their specificity toward a desired sequence. A desirable nucleic acid recognition sequence consists of a nucleotide sequence spanning at least ten, preferably eleven, and more preferably twelve or more bases. The component binding portions (putative or demonstrated) within the nucleotide sequence need not be fully contiguous; they may be interspersed with “spacer” base pairs that need not be directly contacted by the chimeric protein but rather impose proper spacing between the nucleic acid subsites recognized by each module. These sequences should not impart expression to linked genes when introduced into cells in the absence of the engineered DNA-binding protein.

To identify a nucleotide sequence that is recognized by a transcriptional activator protein containing the composite DNA-binding region, preferably recognized with high affinity (dissociation constant 10⁻¹¹ M or lower are especially preferred), several methods can be used. If high-affinity binding sites for individual subdomains of the composite DNA-binding region are already known, then these sequences can be joined with various spacing and orientation and the optimum configuration determined experimentally (see below for methods for determining affinities). Alternatively, high-affinity binding sites for the protein or protein complex can be selected from a large pool of random DNA sequences by adaptation of published methods (Pollock, R. and Treisman, R., 1990, A sensitive method for the determination of protein-DNA binding specificities. Nucl. Acids Res. 18, 6197-6204). Bound sequences are cloned into a plasmid and their precise sequence and affinity for the proteins are determined. From this collection of sequences, individual sequences with desirable characteristics (i.e., maximal affinity for composite protein, minimal affinity for individual subdomains) are selected for use. Alternatively, the collection of sequences is used to derive a consensus sequence that carries the favored base pairs at each position. Such a consensus sequence is synthesized and tested (see below) to confirm that it has an appropriate level of affinity and specificity.

A number of well-characterized assays are available for determining the binding affinity, usually expressed as dissociation constant, for DNA-binding proteins and the cognate DNA sequences to which they bind. These assays usually require the preparation of purified protein and binding site (usually a synthetic oligonucleotide) of known concentration and specific activity. Examples include electrophoretic mobility-shift assays, DNasel protection or “footprinting”, and filter-binding. These assays can also be used to get rough estimates of association and dissociation rate constants. These values may be determined with greater precision using a BlAcore instrument. In this assay, the synthetic oligonucleotide is bound to the assay “chip,” and purified DNA-binding protein is passed through the flow-cell. Binding of the protein to the DNA immobilized on the chip is measured as an increase in refractive index. Once protein is bound at equilibrium, buffer without protein is passed over the chip, and the dissociation of the protein results in a return of the refractive index to baseline value. The rates of association and dissociation are calculated from these curves, and the affinity or dissociation constant is calculated from these rates. Binding rates and affinities for the high affinity composite site may be compared with the values obtained for subsites recognized by each subdomain of the protein. As noted above, the difference in these dissociation constants should be at least two orders of magnitude and preferably three or greater.

For additional examples, information and guidance on designing, mutating, selecting, combining and characterizing DNA binding domains, see, e.g., Pomerantz J L, Wolfe S A, Pabo C O, Structure-based design of a dimeric zinc finger protein Biochemistry 1998 Jan 27;37(4):965-970; Kim J-S and Pabo C O, Getting a Handhold on DNA: Design of Poly-Zinc Finger Proteins with Femtomolar Dissociation Constants, PNAS USA, 1998 Mar 17;95(6):2812-2817; Kim J S, Pabo C O, Transcriptional repression by zinc finger peptides. Exploring the potential for applications in gene therapy. , J Biol Chem 1997 Nov 21;272(47):29795-29800; Greisman H A, Pabo C O , A general strategy for selecting high-affinity zinc finger proteins for diverse DNA target sites, Science 1997 Jan 31;275(5300):657-661; Rebar E.J, Greisman H.A, Pabo C.O, Phage display methods for selecting zinc finger proteins with novel DNA-binding specificities, Methods Enzymol 1996;267:129-149; Pomerantz J.L, Pabo C.O, Sharp P.A, Analysis of homeodomain function by structure-based design of a transcription factor, Proc Natl Acad Sci USA 1995 Oct 10;92(21):9752-9756; Rebar E J, Pabo C O, Zinc finger phage: affinity selection of fingers with new DNA-binding specificities, Science 1994, Feb 4;263:671-673; Choo Y, Sanches-Garcia I, Klug A, In vivo repression by a site-specific DNA-binding protein designed against an oncogenic sequence, Nature 1994, Dec 15;372:642-645; Choo Y, Klug A, Toward a code for the interaction of zinc fingers with DNA: Selection of randomized fingers displayed on phage, PNAS USA, Nov 1994; 91:11163-11167; Wu H, Yang W-P, Barbas C F III, Building zinc fingers by selection: toward a therapeutic application, PNAS USA January 1995; 92:344-348; Jamieson A C, Kim S-H, Wells J A, In Vitro selection of zinc fingers with altered DNA-binding specificity, Biochemistry 1994, 33:5689-5695; International patent applications WO 96/20951, WO 94/18317, WO 96/06166 and WO 95/19431; and U.S. Ser. No. 60/084819.

Ligand Binding Domains

Fusion proteins containing a ligand binding domain for use in practicing this invention can function through one of a variety of molecular mechanisms.

In certain embodiments, the ligand binding domain permits ligand-mediated cross-linking of the fusion protein molecules bearing appropriate ligand binding domains. In these cases, the ligand is at least divalent and functions as a dimerizing agent by binding to the two fusion proteins and forming a cross-linked heterodimeric complex which activates target gene expression. See e.g. WO 94/18317, WO 96/20951, WO 96/06097, WO 97/31898 and WO 96/41865.

In other embodiments, the binding of ligand to fusion protein is thought to result in an allosteric change in the protein leading to the binding of the fusion protein to a target DNA sequence [see e.g. U.S. Pat. No. 5,654,168 and 5,650,298 (tet systems), and WO 93/23431 and WO 98/18925 (RU486-based systems)] or to another protein which binds to the target DNA sequence [see e.g. WO 96/37609 and WO 97/38117 (ecdysone/RXR-based systems)], in either case, modulating target gene expression.

Dimerization-based Systems

In the cross-linking-based dimerization systems the fusion proteins can contain one or more ligand binding domains (in some cases containing two, three or four such domains) and can further contain one or more additional domains, heterologous with respect to the ligand binding domain, including e.g. a DNA binding domain, transcription activation domain, etc.

In general, any ligand/ligand binding domain pair may be used in such systems. For example, ligand binding domains may be derived from an immunophilin such as an FKBP, cyclophilin, FRB domain, hormone receptor protein, antibody, etc., so long as a ligand is known or can be identified for the ligand binding domain.

For the most part, the receptor domains will be at least about 50 amino acids, and fewer than about 350 amino acids, usually fewer than 200 amino acids, either as the natural domain or truncated active portion thereof. Preferably the binding domain will be small (<25 kDa, to allow efficient transfection in viral vectors), monomeric, nonimmunogenic, and should have synthetically accessible, cell permeant, nontoxic ligands as described above.

Preferably the ligand binding domain is for (i.e., binds to) a ligand which is not itself a gene product (i.e., is not a protein), has a molecular weight of less than about 5 kD and preferably less than about 2.5 kD, and is cell permeant. In many cases it will be preferred that the ligand does not have an intrinsic pharmacologic activity or toxicity which interferes with its use as a transcription regulator.

The DNA sequence encoding the ligand binding domain can be subjected to mutagenesis for a variety of reasons. The mutagenized ligand binding domain can provide for higher binding affinity, allow for discrimination by a ligand between the mutant and naturally occurring forms of the ligand binding domain, provide opportunities to design ligand-ligand binding domain pairs, or the like. The change in the ligand binding domain can involve directed changes in amino acids known to be involved in ligand binding or with ligand-dependent conformational changes. Alternatively, one may employ random mutagenesis using combinatorial techniques. In either event, the mutant ligand binding domain can be expressed in an appropriate prokaryotic or eukarotic host and then screened for desired ligand binding or conformational properties. Examples involving FKBP, cyclophilin and FRB domains are disclosed in detail in WO 94/18317, WO 96/06097, WO 97/31898 and WO 96/41865. For instance, one can change Phe36 to Ala and/or Asp37 to Gly or Ala in FKBP12 to accommodate a substituent at positions 9 or 10 of the ligand FK506 or FK520 or analogs, mimics, dimers or other derivatives thereof. In particular, mutant FKBP12 domains which contain Val, Ala, Gly, Met or other small amino acids in place of one or more of Tyr26, Phe36, Asp37, Tyr82 and Phe99 are of particular interest as receptor domains for FK506-type and FK-520-type ligands containing modifications at C9 and/or C10 and their synthetic counterparts (see e.g., WO 97/31898). Illustrative mutations of current interest in FKBP domains also include the following:

TABLE 1
Entries identify the native amino acid by single letter code and sequence
position, followed by the replacement amino acid in the mutant. Thus,
F36V Designates a human FKBP12 sequence in which phenylalanine at
position 36 is replaced by valine. F36V/F99A indicates a double mutation
in which phenylalanine at positions 36 and 99 are replacedby valine and
alanine, respectively.

| | | | |
|---|---|---|---|
| F36A | Y26V | F46A | W59A |
| F36V | Y26S | F48H | H87W |
| F36M | D37A | F48L | H87R |
| F36S | I90A | F48A | F36V/F99A |
| F99A | I91A | E54A/F36V/F99G | F99G |
| F46H | E54K/F36M/F99A | Y26A | F46L |
| V55A | F36M/F99G | | |

Illustrative examples of domains which bind to the FKBP:rapamycin complex (“FRBs”) are those which include an approximately 89-amino acid sequence containing residues 2025-2113 of human FRAP. Another FRAP-derived sequence of interest comprises a 93 amino acid sequence consisting of amino acids 2024-2113. Similar considerations apply to the generation of mutant FRAP-derived domains which bind preferentially to FKBP complexes with rapamycin analogs (rapalogs) containing modifications (i.e., are ‘bumped’) relative to rapamycin in the FRAP-binding portion of the drug. For example, one may obtain preferential binding using rapalogs bearing substituents other than —OMe at the C7 position with FRBs based on the human FRAP FRB peptide sequence but bearing amino acid substitutions for one of more of the residues Tyr2038, Phe2039, Thr2098, Gln2099, Trp2lOl and Asp2102. Exemplary mutations include Y2038H, Y2038L, Y2038V, Y2038A, F2039H, F2039L, F2039A, F2039V, D2102A, T2098A, T2098N, T2098L, and T2098S. Rapalogs bearing substituents other than —OH at C28 and/or substituents other than ═O at C30 may be used to obtain preferential binding to FRAP proteins bearing an amino acid substitution for Glu2032. Exemplary mutations include E2032A and E2032S. Proteins comprising an FRB containing one or more amino acid replacements at the foregoing positions, libraries of proteins or peptides randomized at those positions (i.e., containing various substituted amino acids at those residues), libraries randomizing the entire protein domain, or combinations of these sets of mutants are made using the procedures described above to identify mutant FRAPs that bind preferentially to bumped rapalogs.

Other macrolide binding domains useful in the present invention, including mutants thereof, are described in the art. See, for example, WO96/41865, WO96/13613, WO96/061 11, WO96/061 10, WO96/06097, WO96/12796, WO95/05389, WO95/02684, WO094/18317.

The ability to employ in vitro mutagenesis or combinatorial modifications of sequences encoding proteins allows for the production of libraries of proteins which can be screened for binding affinity for different ligands. For example, one can randomize a sequence of 1 to 5, 5 to 10, or 10 or more codons, at one or more sites in a DNA sequence encoding a binding protein, make an expression construct and introduce the expression construct into a unicellular microorganism, and develop a library of modified sequences. One can then screen the library for binding affinity of the encoded polypeptides to one or more ligands. The best affinity sequences which are compatible with the cells into which they would be introduced can then be used as the ligand binding domain for a given ligand. The ligand may be evaluated with the desired host cells to determine the level of binding of the ligand to endogenous proteins. A binding profile may be determined for each such ligand which compares ligand binding affinity for the modified ligand binding domain to the affinity for endogenous proteins. Those ligands which have the best binding profile could then be used as the ligand. Phage display techniques, as a non-limiting example, can be used in carrying out the foregoing.

In other embodiments, antibody subunits, e.g. heavy or light chain, particularly fragments, more particularly all or part of the variable region, or single chain antibodies, can be used as the ligand binding domain. Antibodies can be prepared against haptens which are pharmaceutically acceptable and the individual antibody subunits screened for binding affinity. cDNA encoding the antibody subunits can be isolated and modified by deletion of the constant region, portions of the variable region, mutagenesis of the variable region, or the like, to obtain a binding protein domain that has the appropriate affinity for the ligand. In this way, almost any physiologically acceptable hapten can be employed as the ligand. Instead of antibody units, natural receptors can be employed, especially where the binding domain is known. In some embodiments of the invention, a fusion protein comprises more than one ligand binding domain. For example, a DNA binding domain can be linked to 2, 3 or 4 or more ligand binding domains. The presence of multiple ligand binding domains means that ligand-mediated cross-linking can recruit multiple fusion proteins containing transcription activation domains to the DNA binding domain-containing fusion protein.

Allostery-based Systems

As mentioned previously, systems for transcription regulation based on ligand-dependent allosteric changes in a chimeric transcription factor are also useful in practicing the subject invention. One such system employs a deletion mutant of the human progesterone receptor which no longer binds progesterone or other endogenous steroids but can be activated by the orally active progesterone antagonist RU486, described, e.g., in Wang et al. (1994) Proc. Natl. Acad. Sci. U.S.A. 91:8180. Activation was demonstrated in cells transplanted into mice using doses of RU486 (5-50 g/kg) considerably below the usual dose for inducing abortion in humans (10 mg/kg). However, the reported induction ratio in culture and in animals was rather low.

Additional Domains and Linkers

Additional domains may be included in the fusion proteins of this invention.

For example, the fusion proteins may contain a nuclear localization sequence (NLS) which provides for the protein to be translocated to the nucleus. A NLS can be located at the N-terminus or the C-terminus of a fusion protein, or can be located between component portions of the fusion protein, so long as the function of fusion protein and its components is disrupted by presence of the NLS. Typically a nuclear localization sequence has a plurality of basic amino acids, referred to as a bipartite basic repeat (reviewed in Garcia-Bustos et al. (1991) Biochimica et Biophysica Acta 1071:83-101). One illustrative NLS is derived from the NLS of the SV40 large T antigen which is comprised of amino acids proline-lysine-lysine-lysine-arginine-lysine-valine (Kalderon et al. (1984) Cell 39:499-509). Another illustrative NLS is derived from a p53 protein. Wild-type p53 contains three C-terminal nuclear localization signals, comprising residues 316-325, 369-375 and 379-384 of p53 (Shaulsky et al. (1990) Mol. Cell. Biol. 10:6565-6577). Other NLSs are described by Shaulsky et al (1990) supra and Shaulsky et al. (1991) Oncogene 6:2056.

To facilitate their detection and/or purification, the fusion proteins may contain peptide portions such as “histidine tags”, a glutathione-S-transferase domain or an “epitope tag” which can be recognized by an antibody.

The intervening distance and relative orientation of the various component domains of the fusion proteins can be varied to optimize their production or performance. The design of the fusion proteins may include one or more “linkers”, comprising peptide sequence (which may be naturally-occurring or not) separating individual component polypeptide sequences. Many examples of linker sequences, their occurrence in nature, their design and their use in fusion proteins are known. See e.g. Huston et al. (1988) PNAS 85:4879; U.S. Pat. No. 5,091,513; and Richardson et al. (1988) Science 240:1648-1652.

Ligands

In various embodiments where a ligand binding domain for the ligand is endogenous to the cells to be engineered, it is often desirable to alter the peptide sequence of the ligand binding domain and to use a ligand which discriminates between the endogenous and engineered ligand binding domains. Such a ligand should bind preferentially to the engineered ligand binding domain relative to a naturally occurring peptide sequence, e.g., from which the modified domain was derived. This approach can avoid untoward intrinsic activities of the ligand. Significant guidance and illustrative examples toward that end are provided in the various references cited herein.

Cross-linking/Dimerization Systems

Any ligand for which a binding protein or ligand binding domain is known or can be identified may be used in combination with such a ligand binding domain in carrying out this invention.

Extensive guidance and examples are provided in WO 94/18317 for ligands and other components useful for cross-linked oligomerization-based systems. Systems based on ligands for an immunophilin such as FKBP, a cyclophilin, and/or FRB domain are of special interest. Illustrative examples of ligand binding domain/ligand pairs that may be used for cross-linking include, but are not limited to: FKBP/FK1012 , FKBP/synthetic divalent FKBP ligands (see WO 96/06097 and WO 97/31898), FRB/rapamycin or analogs thereof:FKBP (see e.g., WO 93/33052, WO 96/41865 and Rivera et al, “A humanized system for pharmacologic control of gene expression”, Nature Medicine 2(9):1028-1032 (1997)), cyclophilin/cyclosporin (see e.g. WO 94/18317), FKBP/FKCsA/cyclophilin (see e.g. Belshaw et al, 1996, PNAS 93:4604-4607), DHFR/methotrexate (see e.g. Licitra et al, 1996, Proc. Natl. Acad. Sci. USA 93:12817-12821), and DNA gyrase/coumermycin (see e.g. Farrar et al, 1996, Nature 383:178-181). Numerous variations and modifications to ligands and ligand binding domains, as well as methodologies for designing, selecting and/or characterizing them, which may be adapted to the present invention are disclosed in the cited references.

Allostery-based Systems

For additional guidance on ligands for other systems which may be adapted to this invention, see e.g. (Gossen and Bujard Proc. Natl. Acad. Sci. U.S.A. 1992 89:5547, and US Patent Nos. 5654168, 5650298, 5589362 and 5464758 (TetR/tetracycline), Wang et al, 1994, Proc. Natl. Acad. Sci. USA 91:8180-8184 (progesterone receptor/RU486), and No et al, 1996, Proc. Natl. Acad. Sci. USA 93:3346-3351 (ecdysone receptor/ecdysone).

Ligands for Conditional Aggregation Domains

A wide variety of ligands, including both naturally occurring and synthetic substances, can be used in this invention to effect disaggregation of the fusion protein molecules. Criteria for selecting a ligand are: (A) physiologic acceptability of the ligand (i.e., the ligand lacks undue toxicity towards the cell or animal for which it is to be used), (B) reasonable therapeutic dosage range, (C) suitability for oral administration (i.e., suitable stability in the gastrointestinal system and absorption into the vascular system), for applications in whole animals, including gene therapy applications, (D) ability to cross cellular and other membranes, as necessary, and (E) reasonable binding affinity for the CAD (for the desired application). Preferably the compound is relatively physiologically inert, but for its affinity for the CAD. The less the ligand binds to native proteins or other materials within the cells to be targeted, the better the response will normally be. Preferably the ligand will be other than a peptide or nucleic acid, and will preferably have a molecular weight of less than about 5000 Daltons, more preferably less than about 1200 Daltons.

In various embodiments where a ligand binding domain for a candidate ligand is endogenous to the cells to be engineered, it is often desirable to alter the peptide sequence of the ligand binding domain and to use a ligand which discriminates between the endogenous and engineered ligand binding domains. Such a ligand should bind preferentially to the engineered ligand binding domain relative to a naturally occurring peptide sequence, e.g., from which the modified domain was derived. This approach can avoid untoward intrinsic activities of the ligand. Significant guidance and illustrative examples toward that end are provided in the various references cited herein.

Substantial structural modification of a ligand for a ligand binding domain is permitted, so long as the modified compound still functions as a ligand for the ligand binding domain of interest, i.e., so long as the compound possesses sufficient binding affinity and specificity to function as disclosed herein. Some of the compounds will be macrocyclics, e.g. macrolides, although linear and branched compounds may be preferred in specific embodiments. Suitable binding affinities will be reflected in Kd values well below 10⁻⁴, preferably below 10⁻⁶, more preferably below about 10⁻⁷, although binding affinities below 10⁻⁹ or 10⁻¹⁰ are possible, and in some cases will be most desirable.

Illustrative examples of ligand binding domain/ligand pairs include retinol binding protein or variants thereof and retinol or derivatives thereof; cyclophilin or variants thereof and cyclosporin or analogs thereof; FKBP or variants thereof and FK506, FK520, rapamycin, analogs thereof or synthetic FKBP ligands. In the case of a ligand binding domain comprising or derived from an immunophilin or cyclophilin, the complex of the ligand with the ligand binding domain will desirably not bind specifically to calcineurin or FRAP. A wide variety of FK506 derivatives and synthetic FKBP ligands are known which do not have observable immunosuppressive activity. Likewise, a variety of rapamycin analogs are known which bind to FKBP but are not immunosuppressive. See e.g. WO 98/02441for non-immunosuppressive rapalogs. Those and other ligands can be used as well, depending on the choice of CAD.

Ligand binding domain/ligand pairs are illustrated by FKBP domains, e.g. F36M FKBP, and FKBP ligands. In general, it is preferred that the ligand bind preferentially to a mutated (i.e., having a peptide sequence not naturally occurring in the cells to be engineered) FKBP relative to wild-type FKBP. Ligands for FKBP proteins, including F36M FKBP, can comprise or be derived from a naturally occurring FKBP ligand such as rapamycin, FK506 or FK520, or a synthetic FKBP ligand, e.g. as disclosed in PCT/US95/10559; Holt, et al., J. Amer. Chem. Soc.,1993, 715, 9925-9938; Holt, et al., Biomed. Chem. Lett., 1993, 4, 315-320; Luengo, et al., Biomed. Chem. Lett., 1993, 4, 321-324; Yamashita, et al., Biomed. Chem. Lett., 1993, 4, 325-328; PCT/US94/01617; PCT/US94/08008. See also EP 0 455 427 Al; EP 0 465 426 Al; U.S. Pat. No. 5,023,26; WO 92/00278; WO 94/18317; WO 97/31898; WO 96/41865; and Van Duyne et al (1991) Science 252, 839.

Target Gene

As used herein, the term “target gene” refers to a gene, whose transcription is stimulated according to the method of the invention. In a preferred embodiment, the gene is integrated in the chromosomal DNA of a cell. A cell comprising a target gene is referred to herein as a “target cell”.

In a preferred embodiment of the invention, the target gene is an endogenous gene. As used herein, the term “endogenous gene” refers to a gene which is naturally present in a cell, in its natural environment, i.e., not a gene which has been introduced into the cell by genetic engineering. The endogenous gene can be any gene having a promoter that is recognized by at least one transcription factor. In a preferred embodiment, the promoter or any regulatory element thereof, of the endogenous gene (“endogenous promoter” and “endogenous regulatory element”, respectively), is recognized by a known, preferably cloned, DNA binding protein, whether it is a transcriptional activator or repressor. Alternatively, if no DNA binding protein is known to interact with a target promoter, it is possible to clone such a factor using techniques well known in the art without undue experimentation, such as screening of expression libraries with at least a portion of the target promoter. Furthermore, the affinity of binding of a DNA binding domain to a target sequence can be improved according to methods known in the art. Such methods comprise, e.g., introducing mutations into the DNA binding domain and screening for mutants having increased DNA binding affinity.

In another embodiment of the invention, the target gene is an endogenous gene, which contains an exogenous target sequence. The exogenous target sequence can be inserted into the endogenous promoter or substitute at least a portion of the endogenous promoter. In preferred embodiments, the exogenous promoter or regulatory element introduced into the endogenous target promoter is recognized by a DNA binding protein, capable of binding with high affinity and specificity to a target sequence. In a preferred embodiment, the DNA binding protein is human. However, the DNA binding protein can be from any other species. For example, the DNA binding protein can be from the yeast GAL4 protein.

The proteins which are expressed, singly or in combination, can involve homing, cytotoxicity, proliferation, immune response, inflammatory response, clotting or dissolving of clots, hormonal regulation, etc. The proteins expressed may be naturally-occurring proteins, mutants of naturally-occurring proteins, unique sequences, or combinations thereof.

Various secreted products include hormones, such as insulin, human growth hormone, glucagon, pituitary releasing factor, ACTH, melanotropin, relaxin, leptin,etc.; growth factors, such as EGF, IGF-1, TGF-alpha, -beta, PDGF, G-CSF, M-CSF, GM-CSF, FGF, erythropoietin, thrombopoietin, megakaryocytic growth factors, nerve growth factors, etc.; proteins which stimulate or inhibit angiogenesis such as angiostatin, endostatin and VEGF and variants thereof; interleukins, such as IL-1 to -15; TNF-alpha and -beta; and enzymes and other factors, such as tissue plasminogen activator, members of the complement cascade, perforins, superoxide dismutase; coagulation-related factors such as antithrombin-III, Factor V, Factor VII, Factor VIIIc, vWF, Factor IX, alpha-anti-trypsin, protein C, and protein S; endorphins, dynorphin, bone morphogenetic protein, CFTR, etc.

The gene can encode a naturally-occurring surface membrane protein. Various such proteins include homing receptors, e.g. L-selectin (Mel-14), hematopoietic cell markers, e.g. CD3, CD4, CD8, B cell receptor, TCR subunits alpha, beta, gamma or delta, CD10, CD19, CD28, CD33, CD38, CD41, etc., receptors, such as the interleukin receptors IL-2R, IL-4R, etc.; receptors for other ligands including the various hormones, growth factors, etc.; receptor antagonists for such receptors and soluble forms of such receptors; channel proteins, for influx or efflux of ions, e.g. H⁺, Ca⁺², K⁺, Na⁺, Cl⁻, etc., and the like; CFTR, tyrosine activation motif, zap-70, etc.

Also, intracellular proteins can be of interest, such as proteins in metabolic pathways, regulatory proteins, steroid receptors, transcription factors, etc., depending upon the nature of the host cell. Some of the proteins indicated above can also serve as intracellular proteins.

In one embodiment, recognition elements for a DNA binding domain of one of the subject fusion proteins are introduced into the host cells such that they are operatively linked to an endogenous target gene, e.g. by homologous recombination with genomic DNA. A variety of suitable approaches s are available. See, e.g., PCT publications WO93/09222, WO95/31560, WO96/29411, WO95/31560 and WO94/12650. This permits ligand-mediated regulation of the transcription of the endogenous gene.

(b) Minimal Promoters.

Minimal promoters which may be incorporated into a target gene construct (or other construct of the invention) may be selected from a wide variety of known sequences, including promoter regions from fos, hCMV, SV40 and IL-2, among many others. Illustrative examples are provided which use a minimal CMV promoter or a minimal IL2 gene promoter (-72 to +45 with respect to the start site; Siebenlist et al., MCB 6:3042-3049, 1986)

(c) DNA recognition sequences.

The choice of recognition sequences to use in the target gene construct is in some cases determined by the nature of the regulatory system to be employed.

Where the target gene construct comprises an endogenous gene with its own regulatory DNA, the recognition sequence is thereby provided by the cells. and the practitioner provides a DNA binding domain which recognizes it.

In other cases, e.g., in ligand-mediated crosslinking systems and systems like the progesterone receptor-based system, a diverse set of DNA binding domain:recognition sequence choices are available to the practitioner.

Recognition sequences for a wide variety of DNA-binding domains are known. DNA recognition sequences for other DNA binding domains may be determined experimentally. In the case of a composite DNA binding domain, DNA recognition sequences can be determined experimentally, or the proteins can be manipulated to direct their specificity toward a desired sequence. A desirable nucleic acid recognition sequence for a composite DNA binding domain consists of a nucleotide sequence spanning at least ten, preferably eleven, more preferably twelve or more, and even more preferably in some cases eighteen bases. The component binding portions (putative or demonstrated) within the nucleotide sequence need not be fully contiguous; they may be interspersed with “spacer” base pairs that need not be directly contacted by the chimeric protein but rather impose proper spacing between the nucleic acid subsites recognized by each module. These sequences should not impart expression to linked genes when introduced into cells in the absence of the engineered DNA-binding protein.

To identify a nucleotide sequence that is recognized by a chimeric protein containing a DNA-binding region, preferably recognized with high affinity (dissociation constant 10⁻¹¹ M or lower are especially preferred), several methods can be used. If high-affinity binding sites for individual subdomains of a composite DNA-binding region are already known, then these sequences can be joined with various spacing and orientation and the optimum configuration determined experimentally (see below for methods for determining affinities). Alternatively, high-affinity binding sites for the protein or protein complex can be selected from a large pool of random DNA sequences by adaptation of published methods (Pollock, R. and Treisman, R., 1990, A sensitive method for the determination of protein-DNA binding specificities. Nucl. Acids Res. 18, 6197-6204). Bound sequences are cloned into a plasmid and their precise sequence and affinity for the proteins are determined. From this collection of sequences, individual sequences with desirable characteristics (i.e., maximal affinity for composite protein, minimal affinity for individual subdomains) are selected for use. Alternatively, the collection of sequences is used to derive a consensus sequence that carries the favored base pairs at each position. Such a consensus sequence is synthesized and tested to confirm that it has an appropriate level of affinity and specificity.

The target gene constructs may contain multiple copies of a DNA recognition sequence. For instance, the constructs may contain 5, 8, 10 or 12 recognition sequences for GAL4 or for ZFHD1.

Design and Assembly of the DNA Constructs

Constructs may be designed in accordance with the principles, illustrative examples and materials and methods disclosed in the patent documents and scientific literature cited herein, with modifications and further exemplification as described. Components of the constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. In the case of DNA constructs encoding fusion proteins, DNA sequences encoding individual domains and sub-domains are joined such that they constitute a single open reading frame encoding a fusion protein capable of being translated in cells or cell lysates into a single polypeptide harboring all component domains. The DNA construct encoding the fusion protein may then be placed into a vector for transducing host cells and permitting the expression of the protein. For biochemical analysis of the encoded chimera, it may be desirable to construct plasmids that direct the expression of the protein in bacteria or in reticulocyte-lysate systems. For use in the production of proteins in mammalian cells, the protein-encoding sequence is introduced into an expression vector that directs expression in these cells. Expression vectors suitable for such uses are well known in the art. Various sorts of such vectors are commercially available.

Introduction of Constructs into Cells

This invention is particularly useful for the engineering of animal cells and in applications involving the use of such engineered animal cells. The animal cells may be insect, worm or mammalian cells. While various mammalian cells may be used, including, by way of example, equine, bovine, ovine, canine, feline, murine, and non-human primate cells, human cells are of particular interest. Across the various species, various types of cells may be used, such as hematopoietic, neural, glial, mesenchymal, cutaneous, mucosal, stromal, muscle (including smooth muscle cells), spleen, reticuloendothelial, epithelial, endothelial, hepatic, kidney, gastrointestinal, pulmonary, fibroblast, and other cell types. Of particular interest are muscle cells (including skeletal, cardiac and other muscle cells), cells of the central and peripheral nervous systems, and hematopoietic cells, which may include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells.

In some instances specific clones or oligoclonal cells may be of interest, where the cells have a particular specificity, such as T cells and B cells having a specific antigen specificity or homing target site specificity.

Constructs encoding the fusion proteins and comprising target genes of this invention can be introduced into the cells as one or more nucleic acid molecules or constructs, in many cases in association with one or more markers to allow for selection of host cells which contain the construct(s). The constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional domain may be isolated, where one or more mutations may be introduced using “primer repair”, ligation, in vitro mutagenesis, etc. as appropriate.

The construct(s) once completed and demonstrated to have the appropriate sequences may then be introduced into a host cell by any convenient means. The constructs may be incorporated into vectors capable of episomal replication (e.g. BPV or EBV vectors) or into vectors designed for integration into the host cells' chromosomes. The constructs may be integrated and packaged into non-replicating, defective viral genomes like Adenovirus, Adeno-associated virus (AAV), or Herpes simplex virus (HSV) or others, including retroviral vectors, for infection or transduction into cells. Alternatively, the construct may be introduced by protoplast fusion, electroporation, biolistics, calcium phosphate transfection, lipofection, microinjection of DNA or the like. The host cells will in some cases be grown and expanded in culture before introduction of the construct(s), followed by the appropriate treatment for introduction of the construct(s) and integration of the construct(s). The cells will then be expanded and screened by virtue of a marker present in the constructs. Various markers which may be used successfully include hprt, neomycin resistance, thymidine kinase, hygromycin resistance, etc., and various cell-surface markers such as Tac, CD8, CD3, Thy1 and the NGF receptor.

In some instances, one may have a target site for homologous recombination, where it is desired that a construct be integrated at a particular locus. For example, one can delete and/or replace an endogenous transcription control element with an exogenous promoter. For homologous recombination, one may generally use either Ω or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding all of the genes, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, and mammalian expression control elements, etc. which may be used to prepare stocks of construct DNAs and for carrying out transfections are well known in the art, and many are commercially available.

Kits

This invention further provides kits useful for the various applications. One such kit contains one or more nucleic acids, each encoding a fusion protein of the invention. The kit may further contain a sample of a ligand for regulating gene expression using these materials.

Uses

Cells engineered in accordance with the invention are used to produce a target protein in vitro. In such applications, the cells are cultured or otherwise maintained until production of the target protein is desired. At that time, the appropriate ligand is added to the culture medium, in an amount sufficient to cause the desired level of target protein production. The protein so produced may be recovered from the medium or from the cells, and may be purified from other components of the cells or medium as desired.

Proteins for commercial and investigational purposes are often produced using mammalian cell lines engineered to express the protein. The use of mammalian cells, rather than bacteria, insect or yeast cells, is indicated where the proper function of the protein requires post-translational modifications not generally performed by non-mammalian cells. Examples of proteins produced commercially this way include, among others, erythropoietin, BMP-2, tissue plasminogen activator, Factor VIII:c, Factor IX, and antibodies. The cost of producing proteins in this fashion is related to the level of expression achieved in the engineered cells. Thus, because the invention described herein can achieve considerably higher expression levels than conventional expression systems, it may reduce the cost of protein production. Toxicity of target protein production can represent a second limitation, preventing cells from growing to high density and/or reducing production levels. Therefore, the ability to tightly control protein expression, as described herein, permits cells to be grown to high density in the absence of protein production. Expression of the target gene can be activated and the protein product subsequently harvested, only after an optimum cell density is reached, or when otherwise desired. The target gene to be regulated can be an endogenous gene, and its expression may be activated or repressed by addition of ligand.

Cells which have been modified ex vivo with the DNA constructs of the invention may be grown in culture under selective conditions and cells which are selected as having the desired construct(s) may then be expanded and further analyzed, using, for example, the polymerase chain reaction for determining the presence of the construct in the host cells and/or assays for the production of the desired gene product(s). Once modified host cells have been identified, they may then be used as planned, e.g. grown in culture for production of protein.

In cases in which the target gene is an endogenous gene of the cells to be engineered, the promoter and/or one or more other regions of the gene can be modified to include a target sequence that is specifically recognized by the DNA binding domain of a fusion protein of this invention so that the endogenous target gene is specifically recognized and regulated in a ligand-dependent manner. Such an embodiment can be useful in situations in which no DNA binding protein is known to specifically bind to a regulatory region of the target gene. Thus, in one embodiment, one or more cells are obtained and genetically engineered in vitro such that a desired control element is inserted, operatively linked to the target gene. Alternatively, the cell is further modified to include a nucleic acid encoding a fusion protein comprising a DNA binding domain which is capable of interacting specifically with the expression control element introduced into the target gene. In other examples of the invention, an endogenous gene is modified in vivo by, e.g., homologous recombination, a technique well known in the art, and described, e.g., in Thomas and Capecchi (1987) Cell 51:503; Mansour et al. (1988) Nature 336:348; and Joyner et al. (1989) Nature 338:153.

The present invention is further illustrated by the following examples which should not be construed as limiting in any way. The contents of all cited references including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning†A Laboratory†Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Construction of Plasmids Encoding Bundled Activation Domains

Transcription factor fusion proteins were expressed from pCGNN (Attar, R. M. & Gilman, M. Z. (1992) Expression cloning of a novel zinc-finger protein that binds to the c-fos serum response element. Mol. Cell. Biol. 12, 2432-2443). Inserts cloned into pCGNN as XbaI-BamHI fragments are transcribed under control of the human CMV enhancer and promoter and are expressed with an amino-terminal epitope tag (a 16-amino acid portion of the Haemophilus influenzae hemagglutinin gene) and nuclear localization sequence from the SV40 large T antigen. Individual components of the transcription factors were synthesized by polymerase chain reaction as fragments containing an XbaI site immediately upstream of the first codon and a SpeI site, an in-frame stop codon, and a BamHI site immediately downstream of the last codon. Fusion proteins comprising multiple component were assembled by stepwise insertion of XbaI-BamHI fragments into SpeI/BamHI-opened vectors. The individual components used and their abbreviations are as follows:

G=yeast Gal4 DNA binding domain, amino acids 1-94

F=human FKBP12, amino acids 1-107

R=FRB domain of human FRAP, amino acids 2025-2113

S=activation domain from the p65 subunit of human NF-kB, amino acids 361-550

V=activation domain from Herpesvirus VP16, amino acids 410-494

L=E. coli lactose repressor, amino acids 46-360

MT=Minimal Tetramerization domain of E. coli lactose repressor, amino acids 324-360

For example, pCGNN-GF2 was made by insertion of the Gal4 DNA binding domain into pCGNN to generate pCGNN-G, followed by the sequential insertion of 2 FKBP domains. PCGNN-L was made inserting the XbaI/BamHI digested PCR fragments of lactose repressor coding sequences (amino acids 46-360) into PCGNN vector. PCGNN-LS was made by inserting p65 activation domain (amino acids 361-550) into Spe1 and BamH1 digested PCGNN-L expression plasmid. PCGNN-GAL4 CB was made by inserting Xba1 and BamHl digested fragments of c-CBL sequences into Spe1 and BamH1 digested PCGNN-GAL4 expression plasmid. PCGNN-MA was made by inserting Xba1 and BamH1 digested DNA fragments containing SH3 domain coding sequences into Xba1/BamH1 digested PCGNN. PCGNN-MAS and PCGNN-MAMTS were made by inserting the S (p65 activation domain) and MTS (minimal tetramerization domain fused to p65 activation domain) respectively into Spe1/BamH1 digested PCGNN-MA vector.

5xGAL4-IL2-SEAP contains 5 GAL4 sites upstream of a minimal IL2 promoter driving expression of the SEAP gene (a gift of J. Morgenstern and S. Ho). The retroviral vector pLH-5xGal4-IL2-SEAP was constructed by cloning the 5xGAL4-IL2-SEAP fragment described above into the vector pLH (Rivera et al, 1996, Nature Medicine 2:1028-1032; Natesan et al, Nature 1997 Nov 27 390:6658 349-50), which also contains the hygromycin B resistance gene driven by the Moloney murine leukemia virus long terminal repeat.

### Example 2

### Generation of Stable Cell Lines

To generate cells containing the pLH-5xGAL4-IL2-SEAP reporter stably integrated, helper-free retrovirus, generated as described (Rivera et al, 1996; Natesan et al, 1997), was used to infect HT1080 cells. Hundreds of hygromycin B (300 mg/ml) resistant clones were pooled (HT1080 B pool) and individual clones screened by transient transfection with PCG-GS. The most responsive clone, HT1080B, was selected for further analysis.

### Example 3

### Transient Transfections

HT1080 cells were grown at 37° C. in MEM medium containing 10% fetal calf serum, non-essential amino acids and penicillin-streptomycin. Twenty-four hours before transfection, approximately 2×10⁵ cells were seeded in each well in a 12-well plate. Cells were transfected using Lipofectamine as recommended (Gibco BRL). Cells in each well received the amounts plasmids indicated in the figure, with or without 400 ng of reporter plasmid, with the total amount of DNA being adjusted to 1.25 ug with pUC1 9. For experiments shown in FIG. 5, 10 ng of plasmid expressing DNA binding domain fusions and increasing amounts of plasmid expressing p65 activation domain fusions were included. After transfection for five hrs, the medium was removed and 1 ml of fresh medium added. 18-24 hrs later, 100 ul medium was removed and assayed for SEAP activity using a Luminescence Spectrometer (Perkin Elmer) at 350 nm excitation and 450 nm emission. Where indicated, 2-5 ul of medium was also assayed for hGH protein as recommended (Nichols Diagnostic).

### Example 4

### Delivery of Bundled Activation Domains to the GAL4 DNA Binding Domain

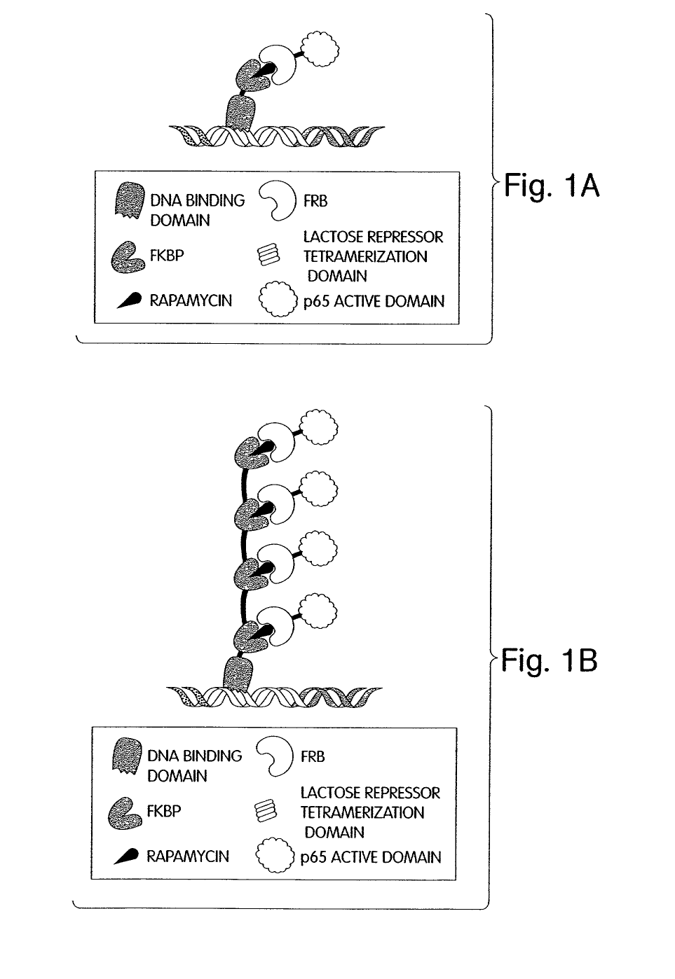

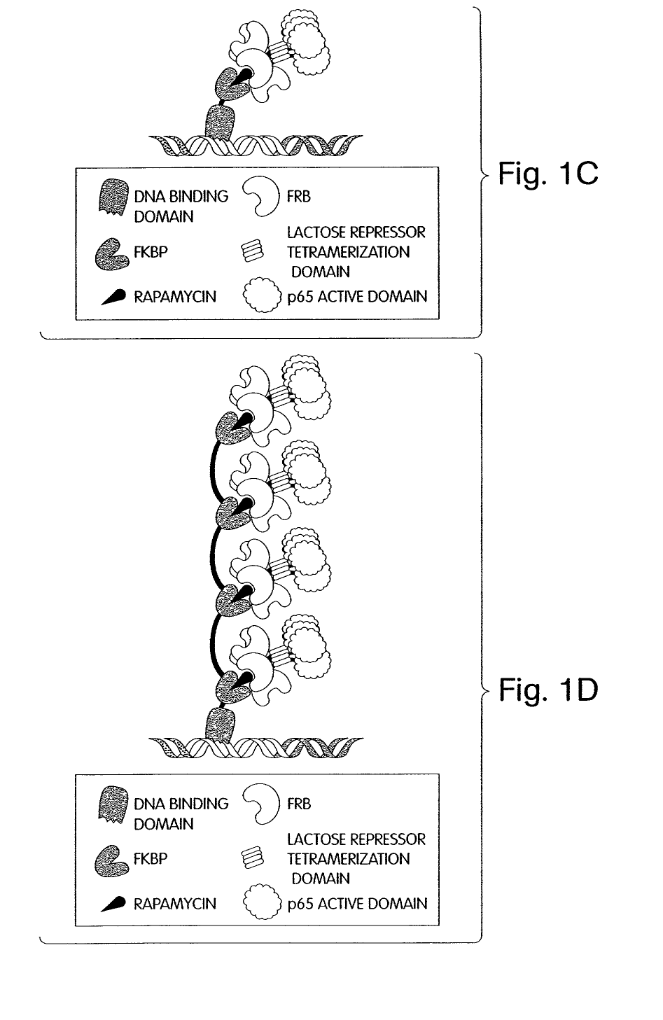

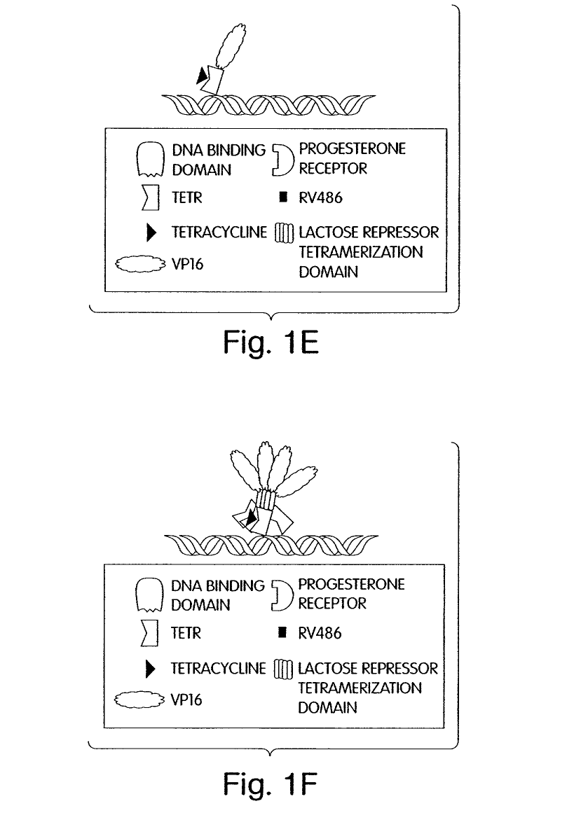

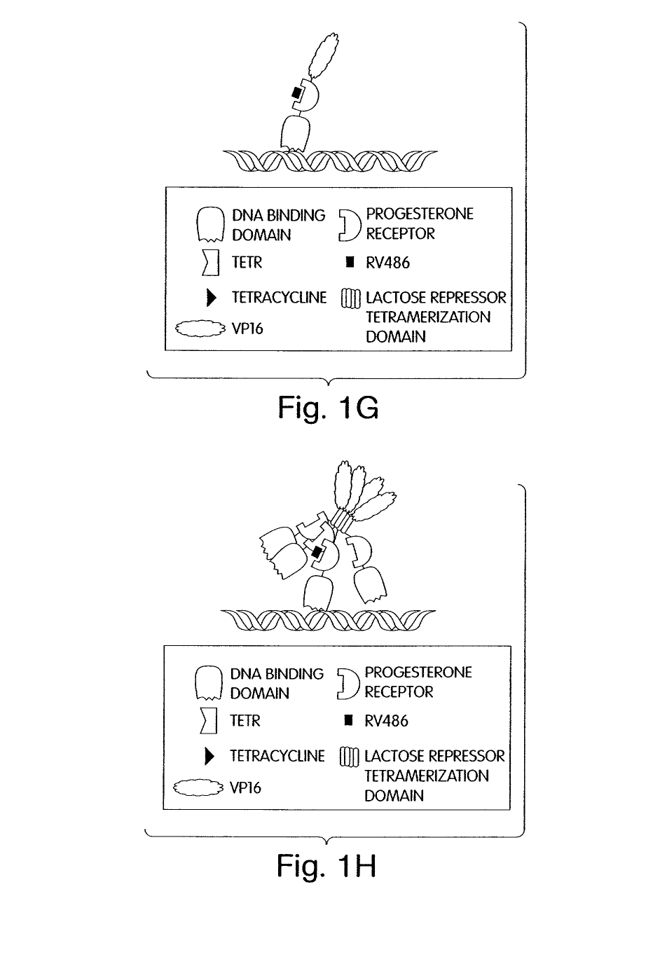

The basic system used for regulated gene expression (FIG. 1A) involves two fusion proteins, one containing a DNA-binding domain (such as GAL4) fused to a single copy of FKBP12 and the other containing a transcription activation domain (such as from the p65 subunit of NF-kB) fused to the FRB domain of FRAP (see e.g., Rivera et al). In the presence of the natural-product rapamycin, which forms a high affinity complex with FKBP and FRB domains, the FRB-p65 fusion protein is efficiently recruited to the GAL4-FKBP fusion protein. This basic system results in the delivery of a maximum of one p65 activation domain per DNA binding domain monomer (FIG. 1A). In this system the number of activation domains delivered to the promoter can be increased by fusing multiple FKBP moieties to GAL4, allowing each DNA binding domain to recruit multiple FRB-p65 activation domain fusions (FIG. 1 B). Because the fusion protein containing the activation domain is expressed separately in this system, it is possible to bundle activation domain fusion proteins and deliver them to FKBP moieties linked to the GAL4 DNA binding domain. For example, the addition of a tetramerization domain present in the E. coli lactose repressor between the FRB and activation domains should generate a fusion protein “bundle” comprising of four activation domains and FRB domains, which in the presence of “dimerizer” can be delivered to each FKBP moiety (FIG. 1C). In the configuration depicted in FIG. 1D rapamycin mediates the recruitment of a tetrameric complex of bundled activation domain fusion proteins to each FKBP of a Gal4-4xFKBP fusion protein, permitting recruitment of up to sixteen p65 activation domains to a single GAL4 monomer. Analogous improvements on allostery-based systems, also based on bundling, are shown in FIGS. 1E-1H.

### Example 5

### Transcriptional Activation is Proportional to the Number of Activation Domains Bound to the Promoter

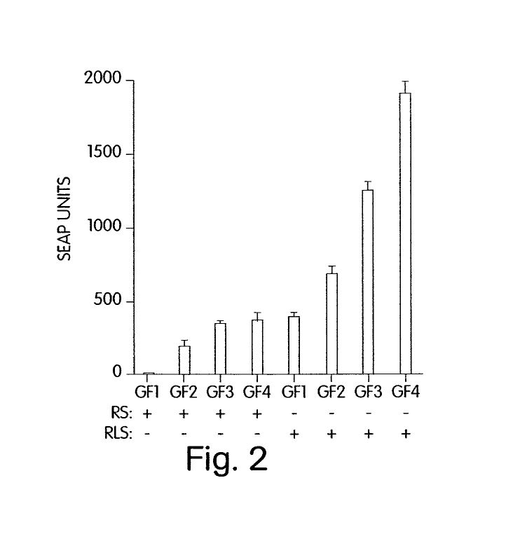

To test how bundled activation domain fusion proteins function in this system, we transfected HT1080 B cells with plasmids expressing various transcription factor fusion proteins and treated the cells with 10 nM rapamycin to deliver the activation domains to the promoter. We observed that when only one RS or RLS fusion protein is delivered to each GAL4 monomer (GF1+RS and GF1+RLS), bundled activation domain fusion proteins induced the reporter gene strongly as compared to the unbundled activation domain fusion proteins. This finding suggests that bundled activation domain fusion proteins, because of their ability to deliver more activation domains to the promoter, function as highly potent inducers of transcription. Furthermore, our studies using various combinations of DNA binding fusion proteins and activation domain fusion proteins revealed that the level of reporter gene expression is roughly linear with the number of activation domains that can be delivered to a single GAL4 monomer bound to its promoter (FIG. 2A).

The RLS fusion protein is capable of delivering four times more p65 activation domain to the promoter than its unbundled counterpart, RS. In theory, FRB fusion protein containing four tandemly reiterated p65 activation domain (RS4) should deliver same number of activation domains to the promoter as RLS and therefore should have similar transactivation capacity. To examine whether RS4 can function in a manner similar to RLS in the rapamycin regulated gene expression system , we transfected expression plasmids encoding the DNA binding receptor, GF1, together with RS4 or RLS fusion proteins into HT1080 B cells and analyzed the expression of the integrated reporter gene by adding 10 nM rapamycin to the medium. We found that rapamycin induced the reporter gene strongly in cells expressing the GF1 and RLS but not the GF1 and RS4 combination of fusion proteins, indicating that the reiterated p65 activation domains are weak inducers of transcription in the dimerizer system. In contrast, rapamycin was able to induce reporter gene expression in the presence of the GF3 and RS4 combination of fusion proteins, albiet at much lower levels than the GF1/RLS combination of proteins. Without being limited to a particular theory, GF3 fusion proteins should recruit three times more activation domains to the promoter than GF1. The finding that RS4 fusion protein can induce transcriptional activation much more strongly when tethered to GF3 as compared to GF1, suggests that when the concentration of activation domain fusion protein is very low, more activation domains can be recruited to the promoter by increasing the number of FKBP moieties fused to the GAL4 DNA binding domain. A western blot analysis of the intracellular levels of the transfected proteins revealed that the amount of RS4 in the cell is below the level of detection, which may explain why it acts as a poor inducer of transcription. These observations strongly suggest that the bundling strategy, unlike reiteration, generates highly potent activation domains that are less toxic to cells.

### Example 6

### Activation of Transcription Using a Minimal Tetramerization Domain and Synergizing Activation Domains

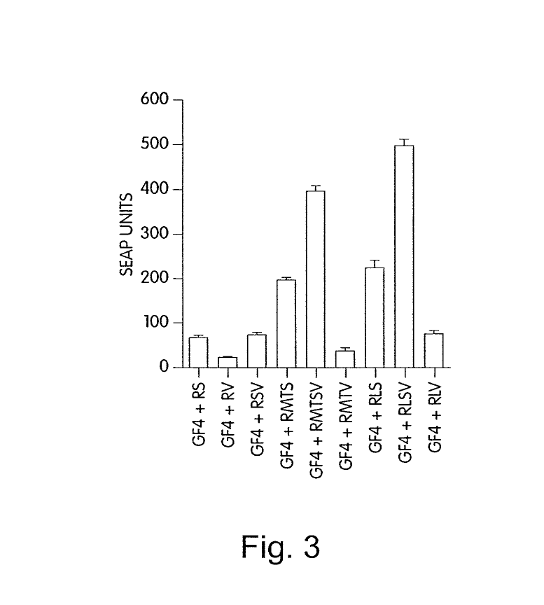

The experiments described used the lactose repressor (minus its DNA binding domain) as the bundling domain in fusion proteins also containing the FRB and activation domains. In addition to the tetramerization domain, this portion of lactose repressor contains the lactose binding domain and the flanking linker regions. To determine whether the tetramerization domain of lactose repressor alone is sufficient for bundling fusion proteins, we made an expression plasmid, RMTS, in which the lactose repressor coding sequences (amino acids 46-360) in the RLS fusion protein was replaced with a thirty-six amino acid region between amino acids 324 and 360 containing the tetramerization domain and a portion of upstream linker region (MT). We have found that combination of p65 and VP16 activation domains when fused to GAL4 DNA binding domain synergistically induced GAL responsive genes. To examine whether they behave similarly when bundled together using the minimal lactose repressor minimal tetramerization domain, we generated two additional plasmids, RMTSV and RMTV in which the VP16 activation domain (amino acids 419-490) was fused to RMTS or RMT respectively. We then co-transfected plasmids expressing appropriate combinations of fusion proteins (FIG. 3) into HT1080 B cells carrying a stably integrated GAL4 responsive reporter gene and treated the cells with rapamycin to stimulate target gene expression. We observed that in cells expressing GF4/RMTSV and GF4/RMTS combination of fusion proteins, rapamycin induced the reporter gene expression to roughly six and three fold higher than GF4/RS combination of fusion proteins. In cells expressing GF4/RMTV or GF4/RSV combinations of fusion proteins, rapamycin induced the reporter gene only marginally higher than the levels induced by GF4/RS fusion proteins (FIG. 3). Although the fold induction of reporter gene expression by GF4/RMTS and GF4/RMTSV is slightly lower than GF4/RLS and GF4/RLSV, three and six fold compared to four and eight fold respectively (see FIG. 2A), strong stimulation of gene expression by the activation domain fusion proteins containing the lactose repressor minimal tetramerization domain suggest that the minimal tetramerization domain is sufficient to bundle fusion proteins.

### Example 7

### Bundling Reduces the Threshold Number of Activators Required to Induce Peak Levels of Gene Expression

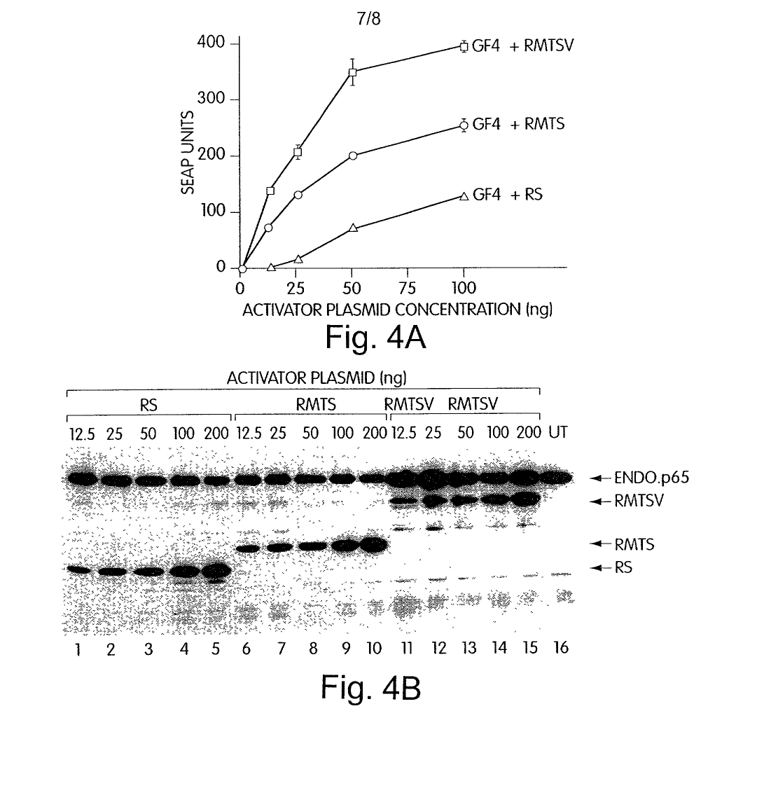

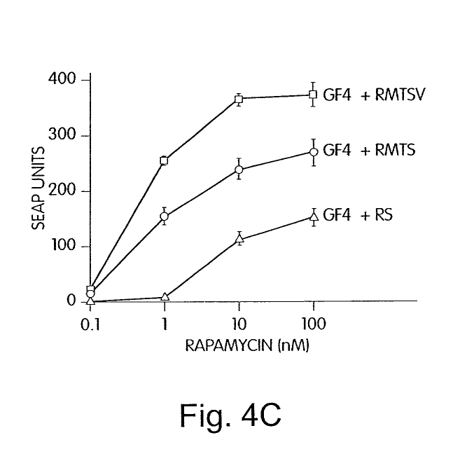

If the strong stimulation of gene expression induced by the bundled fusion proteins containing p65 activation domains is simply due to their ability to deliver more activation domains to the promoter, a lower level of fusion protein containing the activation domain should be sufficient in the case of bundling, as compared to unbundled activation domains, to strongly stimulate reporter gene expression. In the dimerizer system, the number of reconstituted activators formed can be controlled either by adjusting the amount of activation domain fusion proteins or by varying the amount of rapamycin added to the medium. We have employed both of these complementary approaches to address the question of whether bundling of activation domains reduces the threshold amount of activators required for robust expression of the reporter gene. In the first approach, varying amounts of bundled activation domains, RMTS and RMTSV, or their unbundled counterpart, RS, were expressed in HT1080 B cells together with a fixed amount of GF4, the DNA binding receptor (FIG. 4A). The activators were reconstituted by the addition of 10 nM rapamycin to the medium. The level of recombinant proteins expressed in the transfected cells was determined by western blot analysis (FIG. 4B). At the lowest level of activation domains expressed, rapamycin failed to induce transcription of the reporter gene in cells expressing the GF4+RS combination of fusion proteins. However, we observed robust activation of reporter gene expression in cells containing the GF4+RMTS or RMTSV combination of fusion proteins. When the activation domain fusion proteins were present at high levels, rapamycin induced reporter gene expression to approximately four- and two-fold higher levels in cells containing the GF4+RMTSV and GF4+RMTS combination of fusion proteins, respectively, as compared to GF4+RS fusion proteins. Indeed, the level of reporter gene expression induced by the lowest amounts of RMTSV exceeded the level stimulated by the highest amount of RS fusion proteins in the cell (FIG. 4A). These observations suggest that peak levels of reporter gene expression can be achieved with fewer reconstituted activators containing bundled activation domains than with their unbundled counterparts.

In the second complementary approach, we transfected HT1080 B cells with a fixed amount of the expression plasmids used in FIG. 4B and induced the reconstitution of the activators by adding varying amounts of rapamycin to the medium. In the presence of the GF4 DNA binding receptor, both RMTSV and RMTS fusion proteins induced the reporter gene expression robustly at 1 nM rapamycin in the medium. At this concentration of rapamycin in the medium, the GF4+RS combination of fusion proteins failed to induce the reporter gene significantly above background levels. In all cases, we observed peak levels of reporter gene expression in the presence of 10 nM rapamycin in the medium (FIG. 4B). Collectively, the finding that relatively low numbers of activators containing multiple bundled activation domains are sufficient to strongly induce gene expression suggests that the threshold amount of activators required for peak levels of gene expression can be significantly lowered by increasing the potency of activators.

### Example 8

### Regulated Activity of a Transcription Factor Containing a Conditional Aggregation Domain

The chimeric transcription factor ZFHD-p65 consists of the chimeric DNA binding domain, ZFHD1 (Pomerantz et al., Science 267:93-96, 1995)) fused to a transcriptional activation domain from the p65 subunit of NF-kB (Rivera et al., Nat. Med 2:1028-1032, 1996). Transient transfection of the construct into HT1080 cells along with a secreted alkaline phosphatase (SEAP) reporter gene driven by binding sites for ZFHD1 (Rivera et al., Nat. Med 2:1028-1032, 1996) results in the activation of transcription, as measured by the presence of SEAP activity in the culture supernatant. To determine whether the activity of the transcription factor could be made to be dependent on a monomeric ligand, 6 copies of F(36M) were fused to the amino- or carboxy-terminus of the ZFHD-p65 transcription factor. In the absence of the monomeric ligand, FK506, the activity of the transcription factor is repressed. Treatment of cells with increasing concentration of monomer leads to an increase in the activity of the transcription factor, which peaks at 1 uM. These results suggest that fusion of F(36M) domains to a transcription factor results in its sequestration in an inactive oligomeric complex and that interaction with monomeric ligand results in the release of an active transcription factor.

1. A method for producing a desired protein which comprises:
(a) providing cells containing a recombinant nucleic acid encoding at least one fusion protein which can bind to a selected ligand, wherein the fusion protein comprises a ligand binding domain and a DNA binding domain, and in the presence of such a ligand the cells express a gene operably linked to regulatory DNA to which said DNA binding domain binds;
(b) exposing the cells to the ligand in an amount sufficient for production of the encoded protein; and
(c) recovering the protein so produced from the cells.

2. The method of claim 1 which further comprises a recombinant nucleic acid encoding a second fusion protein which binds to the selected ligand, wherein the second fusion protein comprises a ligand binding domain and a transcription activation domain.

3. The method of claim 1 wherein the fusion protein further comprises a transcription activation domain.

4. The method of claim 1 or 2 wherein the fusion protein further comprises a bundling domain.

5. The method of claim 1 or 2 wherein the ligand binding domain is derived from an immunophilin.

6. The method of claim 5 wherein the ligand binding domain binds a ligand that is or is derived from FK506, FK520, rapamycin or cyclosporin A.

7. The method of claim 1 wherein the ligand binding domain is derived from a steroid hormone binding domain.

8. The method of claim 7 wherein the ligand binding domain is derived from the progesterone receptor.

9. The method of claim 1 wherein the DNA binding domain binds to an expression control sequence of an endogenous gene.

10. The method of claim 1 or 9 wherein the DNA binding domain is a composite DNA binding domain.

11. The method of claim 2 or 3 wherein the transcription activation domain is or is derived from the p65 domain of NF-KB.

12. The method of claim 2 or 3 wherein the transcription activation domain comprises two or more activation units that are mutually heterologous.

13. The method of claim 2 or 3 wherein the transcription activation domain comprises at least one synergizing domain.

14. A method for producing a desired protein which comprises:
(a) providing cells containing recombinant nucleic acids encoding two fusion proteins which self-aggregate in the absence of ligand, wherein:
(i) the first fusion protein comprises a conditional aggregation domain which binds to a selected ligand and a transcription activation domain, and
(ii) the second fusion protein comprising a conditional aggregation domain which binds to a selected and a DNA binding domain, and
(iii) in the absence of ligand, the cells express a gene operably linked to regulatory DNA to which said DNA binding domain binds;
(b) expanding the cells in the presence of ligand in an amount sufficient for repression of the gene;
(c) removing the ligand to induce production of the encoded protein; and
(d) recovering the protein so produced from the cells.

15. The method of claim 14 wherein the first fusion protein further comprises a bundling domain.

16. The method of claim 14 wherein the conditional aggregation domain is derived from an immunophilin.

17. The method of claim 16 wherein the conditional aggregation domain binds a ligand that is or is derived from FK506, FK520, rapamycin or cyclosporin A.

18. The method of claim 14 wherein the DNA binding domain binds to an expression control sequence of an endogenous gene.

19. The method of claim 14 wherein the DNA binding domain is a composite DNA binding domain.

20. The method of claim 14 wherein the transcription activation domain is or is derived from the p65 domain of NF-KB.

21. The method of claim 14 wherein the transcription activation domain comprises two or more activation units that are mutually heterologous.

22. The method of claim 14 wherein the transcription activation domain comprises at least one synergizing domain.
